(19) United States
(12) Patent Application Publication
(10) Pub. No.: US 2022/0042048 A1
(43) Pub. Date: Feb. 10, 2022

(54) NONVIRAL GENERATION OF GENOME EDITED CHIMERIC ANTIGEN RECEPTOR T CELLS

(71) Applicant: Wisconsin Alumni Research Foundation, Madison, WI (US)

(72) Inventors: Krishanu Saha, Middleton, WI (US); Christian Matthew Capitini, Madison, WI (US); Katherine Paige Mueller, Madison, WI (US); Nicole Jenine Piscopo, Madison, WI (US); Amritava Das, Madison, WI (US); Matthew Hull Forsberg, Madison, WI (US); Louise Armie Saraspe, Madison, WI (US)

(21) Appl. No.: 17/407,606

(22) Filed: Aug. 20, 2021

Related U.S. Application Data
(63) Continuation-in-part of application No. PCT/US2021/019806, filed on Feb. 26, 2021.
(60) Provisional application No. 62/982,847, filed on Feb. 28, 2020.

(51) Int. Cl. C12N 15/90; C12N 9/22; C12N 15/11; C12N 5/0783; A61K 35/17

(52) U.S. Cl. CPC C12N 15/907; C12N 9/22; C12N 15/11; C12N 2310/20; A61K 35/17; C12N 2501/599; C12N 2800/80; C12N 5/0636

(57) ABSTRACT

Described herein are non-viral, ex vivo methods of site-specifically inserting a transgene containing a chimeric antigen receptor (CAR) gene into a T cell genome by introducing into a population of unmodified T cells a Cas9 ribonucleoprotein (RNP) and a non-viral double-stranded homology-directed repair (HDR) template, to provide genome-edited T cells. The Cas9 ribonucleoprotein includes a Cas9 protein and a guide RNA that directs double stranded DNA cleavage of a cleavage site in a T cell expressed gene. The non-viral double-stranded HDR template comprises the synthetic DNA sequence flanked by homology arms that are complementary to sequences on both sides of the cleavage site in the T cell expressed gene. The transgene is specifically integrated into the cleavage site of the T cell expressed gene created by the Cas9 RNP in the genome-edited T cells, and the cells are then cultured.

### CROSS-REFERENCE TO RELATED APPLICATIONS

This application is a bypass continuation in part application of PCT/US2021/019806, filed on Feb. 26, 2021, which claims priority to U.S. Provisional Application 62/982,847 filed on Feb. 28, 2020, which are incorporated herein by reference in their entirety.

### STATEMENT REGARDING FEDERALLY SPONSORED RESEARCH & DEVELOPMENT

This invention was made with government support under GM119644 and CA014520 awarded by the National Institutes of Health and under 1645123 and EEC1648035 awarded by the National Science Foundation. The government has certain rights in the invention.

### FIELD OF THE DISCLOSURE

The present disclosure is related to method of preparing genome-edited T cells, particular chimeric antigen receptor (CAR) T cells.

### BACKGROUND

Immunotherapy treatments using T cells expressing a chimeric antigen receptor (CAR T cells) targeted against tumor-associated antigens can provide complete remission for patients afflicted by cancer. Currently, there are over 850 clinical trials underway around the world with CAR T cell immunotherapies, and nearly all of them require the use of viral vectors to deliver the CAR gene into T cells. The use of viral vectors for CAR T cell manufacturing constitutes a bottleneck in the supply chain for biomanufacturing and can be problematic due to (1) batch-to-batch variability, (2) use of xenogeneic components during manufacturing of viral vectors, and (3) the high random integration of viral elements into the human genome. The poorly specified integration of the CAR transgene can lead to heterogeneous expression that can be readily silenced, in part by host cell recognition of viral genetic elements.

Methods to generate CAR T cells generally involve viral vectors, transposons or transient transfection. Autologous CAR T cells are traditionally generated using lentiviruses or retroviruses. They can also be generated using transposon-based systems. All of these systems randomly integrate the CAR transgene throughout the human genome. More recently, transfection with mRNA encoding the CAR has also been reported, however the limited half-life of mRNA ultimately does not provide a durable CAR therapy past a few days to weeks.

Genome editing has been used to generate CAR T cells with a site-specific integration of the CAR, however these methods rely on transduction of the T cells with AAVs. AAV6 has been used to deliver the homology directed repair template that encodes the CAR. This was recently demonstrated for a CD19 CAR appropriate for treatment of hematologic malignancies, but not solid tumors. To date, methods to generate CAR T cells have shown limited to no activity in solid tumors. What is needed are new methods for generating genetically modified T cells, such as CAR T cells, that would lead to measurable efficacy against either hematologic malignancies or solid tumors.

### BRIEF SUMMARY

In one aspect, an ex vivo, non-viral method of site-specifically inserting a transgene containing a chimeric antigen receptor (CAR) gene into a T cell expressed gene to generate CAR T cells, comprising
preparing a non-viral double-stranded homology-directed repair (HDR) template comprising the transgene flanked by homology arms that are complementary to sequences on both sides of a cleavage site in the T cell expressed gene,
introducing into a population of unmodified T cells a Cas9 ribonucleoprotein (RNP) and the double-stranded HDR template, to provide the CAR T cells

wherein the Cas9 RNP comprises a Cas9 protein and a guide RNA that directs double stranded DNA cleavage of a cleavage site in the T cell expressed gene,

wherein the non-viral double-stranded HDR template contains the transgene sequence flanked by homology arms that are complementary to sequences on both sides of the cleavage site in the T cell expressed gene, and

wherein the transgene is specifically integrated into the cleavage site of the T cell expressed gene locus created by the Cas9 RNP in the CAR T cells, and
culturing the CAR T cells in xeno-free medium to provide a cultured population of CAR T cells having the transgene specifically integrated in the T cell expressed gene,
wherein, in the cultured population of CAR T cells, an endogenous promoter of the T cell expressed gene drives expression of the transgene, or wherein the transgene includes a promoter that drives expression of the transgene, and
wherein the CAR gene encodes a fusion protein comprising of one or more antigen-specific extracellular domains coupled to an intracellular domain by a transmembrane domain.

In another aspect, a non-viral produced CAR T cell has a genome having a CAR sequence specifically integrated into a T cell expressed gene, wherein the T cell is enriched for the CD62L and/or CD45RA markers indicative of naïve and stem cell memory phenotypes compared to viral-produced control CAR T cells.

In another aspect, a non-viral produced CAR T cell has a genome having a CAR sequence specifically integrated into a T cell expressed gene, wherein the T cell has reduced expression of TIM3 and/or LAG3 markers of T cell exhaustion compared to viral-produced control CAR T cells.

In another aspect, a method of treating a subject comprises administering any of the foregoing CART cells to a subject in need of adoptive T cell therapy.

Also included is a plasmid of SEQ ID NO: 1.

### BRIEF DESCRIPTION OF THE DRAWINGS

FIGS. 1a-1k show nonviral CRISPR-CAR T cells are efficiently manufactured in 9 days and exhibit decreased detrimental signaling and exhaustion before encountering their target antigen. FIG. 1a is a schematic showing the CAR genetic construct and nonviral strategy to insert the CAR into the first exon of the human TRAC locus. The seed sequence of the gRNA is identified and the protospacer adjacent motif (PAM) for SpyCas9 is underlined (SEQ ID NOS. 2 and 3). LHA: left homology arm, SA: splice acceptor, 2A: self-cleaving peptide, pA: rabbit β-globin polyA terminator. FIG. 1b is a summary of manufacturing schedule and analyses for all cell products. RV-CAR, donor-matched CAR T cell product generated by retroviral transduction with the same third generation anti-GD2 CAR shown in a; NV-mCh, donor-matched control T cell product manufactured nonvirally as in FIG. 1a but with an mCherry fluorescent protein instead of the CAR. FIG. 1c shows representative density flow cytometry plots for transgene and TCR surface protein levels on the manufactured cell products. Y-axis shows CAR or mCherry transgene levels and X-axis shows TCR levels on day 7 post isolation (day 5 post-electroporation for NV-CAR and NV-mCh, and day 4 post viral transfection for control RV-CAR). Boxes show populations selected for downstream analysis in FIG. 1d-f. FIG. 1d shows histograms show CAR expression for the three test groups. Boxplots show the percentage of CAR positive cells in each sample, and mean fluorescence intensity (MFI) values for the CAR expression levels, respectively. NV-CAR N=31; RV-CAR N=39; NV-mCh N=27. FIG. 1e shows histograms show TCR expression on the three test groups. Boxplots show the percentage of CAR positive cells in each sample. NV-CAR N=31; RV-CAR N=39; NV-mCh N=27. FIG. 1f shows histograms show CD62L expression for the three test groups. Boxplots show mean fluorescence intensity (MFI) for CD62L expression. NV-CAR N=31; RV-CAR N=39; NV-mCh N=27. Replicates from 97 samples across 4 separate donors. FIG. 1g shows in-out PCR indicates proper on-target genomic integration of the CAR transgene in NV-CAR cells. Primer locations are shown in a by arrows upstream of the LHA and within the CD28 sequence of the CAR. Untransf., untransfected donor-matched T cells; NTC=non-template control. FIG. 1h shows a Manhattan plot of CHANGE-seq-detected on- and off-target sites organized by chromosomal position with bar heights representing CHANGE-seq read count. The on-target site is indicated with the arrow. FIG. li shows visualization of sites detected by CHANGE-seq. The intended target sequence (SEQ ID NO: 4) is shown in the top line. Cleaved sites (on- and off-target) are shown below and are ordered top to bottom by CHANGE-seq read count, with mismatches to the intended target sequence indicated. Insertions are shown in smaller lettering between genomic positions, deletions are shown by (-). FIG. 1j shows UMAP projections as in j showing only cells for which transgene-positive cells were detected. Transgene-positive cells cluster similarly for both NV-CAR and RV-CAR T cells, but not NV-mCh T cells. FIG. 1k.1, k.2 and k.3, show enrichment of Reactome pathway gene signatures (rows) in the transgene-positive cells from donors 1 and 2. NES, Normalized Enrichment Score. At right, representative gene set enrichment analysis (GSEA) plot of a signature within CAR-positive T cells from a RV-CAR sample, where genes differentially expressed in CAR-positive RV-CAR cells versus CAR-positive NV-CAR cells from donor 1 are listed and ranked. FDR <0.001 for each comparison, by gene-set permutation test. Below the GSEA plot is a heatmap representing transcripts with significant differential expression. Rows represent adjusted p-value using Bonferroni correction for all features in the dataset. FIG. 11 shows cytokine production from conditioned media taken from T cell products at the end of manufacturing (pre-antigen exposure). Values are pooled from all 4 donors. NV-CAR, N=24; RV-CAR, N=33; NV-mCherry N=22. * indicates p<=0.05; ** indicates p<=0.01; *** indicates p<=0.001; **** indicates p<=0.0001.

FIGS. 2a-i show nonviral CRISPR-CAR T cells exhibit a robust cytotoxic response to target antigen-positive tumor cells in vitro and induce tumor regression in vivo with a reduced exhaustion phenotype. FIG. 2a shows cytokine production in conditioned media after a 24 hour co-culture of manufactured T cell products with the target GD2-antigen on CHLA20 neuroblastoma cells. Values are pooled from 2 donors. NV-CAR N=8; RV-CAR (green) N=5; NV-mCh N=8. FIG. 2b shows IncuCyte in vitro assay of T cell potency, averaged across donors. AnnexinV was added as a marker of cell death; y-axis shows GFP-positive cancer cells in each well of a 96-well plate. The ratio of T cells to cancer cells is 5:1. The consistent decrease in CHLA20 cells after 15 hours indicates high potency of both NV-CAR and RV-CAR T cells. NV-CAR (blue) N=12; RV-CAR N=12; CHLA20 neuroblastoma alone N=9. FIG. 2c shows a UMAP projections as in c showing only cells for which transgene was detected. Transgene-positive cells cluster similarly for both NV-CAR and RV-CAR T cells, but not for NV-mCh T cells. FIGS. 2d.1, d.2, and d.3 show enrichment of Reactome pathway gene signatures (rows) in the transgene-positive cells from donors 1 and 2 after co-culture with GD2-positive CHLA20 cancer cells. NES, Normalized Enrichment Score. At right, representative GSEA showing differential cytotoxicity signature of NV-CAR/NV-mCh paired samples for two donors, and NV-CAR/RV-CAR samples. NV-CAR T cells show significant upregulation of cytotoxicity markers relative to NV-mCh control cells after GD2 antigen exposure, while NV-CAR and RV-CAR T cells show no significant difference in activation signature upon GD2 antigen stimulation. FDR<0.001 for each comparison, by gene-set permutation test. Bottom GSEA plot is a heatmap representing transcripts with significant differential expression. Rows represent adjusted p-value using Bonferroni correction for all features in the dataset. FIG. 2e shows a schematic of the in vivo mouse dosing strategy using NSG mice harboring GD2-positive CHLA20 neuroblastoma tumors. FIG. 2f shows representative IVIS images of NSG mice with CHLA20 tumors that were treated with either 10 million NV-CAR, RV-CAR, or NV-mCh T cells. FIG. 2g shows Kaplan-Meyer survival curve for mice. NV-CAR N=10; RV-CAR N=8; NV-mCh N=7. FIG. 2i shows box plots on the amount of human T cells present in mouse spleens, as measured by the presence of human CD45 using flow cytometry, and the percentage of those cells in the spleen that were CAR-positive. FIG. 2h shows histograms showing the expression levels of PD-1 and TIM-3 on the human CD45+ cells in the mouse spleens. *indicates p<=0.05; ** indicates p<=0.01; *** indicates p<=0.001; **** indicates p<=0.0001.

FIG. 3a-e shows pre-antigen exposure characterization of NV-CART cells. FIG. 3a shows left, viability of cells throughout the manufacturing timeline, pooled for all 4 donors. Right, cell counts throughout the manufacture calendar, pooled for all 4 donors. NV-CAR N=36; RV-CAR N=27; NV-mCh N=25. FIG. 3b shows left, Percent of CAR+ cells as measured by flow cytometry when electroporated on day 2 or day 3 post-isolation. Right, Percent of cells with TCR knockout as measured by flow cytometry when electroporated on day 2 or day 3 post-isolation. All groups, N=3. FIG. 3c shows the level of TCR disruption in NV-CAR and NV-mCh T cells measured by both TCR surface expression by flow cytometry (right) and presence of indels at the TRAC locus (left). NV-CAR N=10, NV-mCh N=8. FIG. 3d shows percent of cells with indels at the TRAC locus in both NV-CAR and NV-mCh conditions. NV-CA N=10; NV-mCh N=8, both for one donor. FIG. 3e shows in-out PCR confirming NV-CAR insertion, full gel from FIG. 1g shown. PCR was optimized to minimize off-target amplification which occurs only for fragments <1 kb across the genome. N=3 for all samples from one donor. Untransf., donor matched untransfected control T cells; NTC=non-template control.

FIG. 4a-c show single cell transcriptomic characterization across eleven samples shows distinct transcriptional signatures associated with CAR expression but not mCherry expression, both before and after antigen exposure. FIG. 4a shows a UMAP projection of single cell RNA-seq data showing cells across all eleven samples and two donors, both pre-and post-antigen exposure. N=69,017 single cells. FIG. 4 b shows a UMAP projection as in FIG. 4a, separated to show clustering of transgene positive cells prior to antigen exposure (left) and after 24 hours of in vitro exposure to GD2+ CHLA20 neuroblastoma. FIG. 4c shows a UMAP projection as in FIG. 4a, showing transgene positive cells for each individual sample. CAR-positive cells from NV-CAR and RV-CAR groups consistently cluster regardless of the presence of GD2 antigen, while NV-mCh cells do not, suggesting a distinct transcriptional profile associated with CAR signaling.

FIG. 5 shows a novel plasmid used to generate CAR HDR template via PCR. The PCR primers were designed to amplify the following: TRAC LHA-SA-2A-14g2a-hinge-CD28-OX40-zeta chain-rb_glob_PA_terminator-TRAC RHA. LHA: left homology arm, SA: splice acceptor: 2A: self-cleaving peptide, rb_glob_PA_terminator: rabbit beta globin polyA terminator. One example is shown here, but any synthetic gene sequence could be inserted between the homology arms.

FIG. 6 shows representative images of NV-CRISPR CAR T cells post-editing.

FIG. 7 shows a flow cytometry plot with representative gene editing. TCR expression is shown on the X axis, and CAR expression is on the Y axis, with 94% TCR knockout and 46% CAR knockin.

FIG. 8 shows average gene editing efficiency across 20 replicates per cell type. 20 replicate NV-CAR and NV-mCherry editing experiments yielded an average knockin efficiency of 35% in both conditions, as measured by flow cytometry. Unedited controls show no non-specific staining.

The above-described and other features will be appreciated and understood by those skilled in the art from the following detailed description, drawings, and appended claims.

### DETAILED DESCRIPTION

Described herein are methods to generate genome edited T cells such as CAR T cells using site-specific genome editing where the editing machinery consists only of proteins and nucleic acids without any viral vectors. In an aspect, demonstrated herein is CRISPR-Cas9 mediated genomic insertion of CAR transgenes into the T cell receptor alpha constant, TRAC, locus in primary human T cells collected from healthy donors. These cells, termed nonviral-(NV)-TRAC-CAR T cells, exhibit proper TRAC-specific integration of the CAR transgene, robust gene expression of the CAR mRNA, and translated CAR proteins on the T cell surface. The NV TRAC-CAR T cells potently upregulate cytotoxic transcriptional programs and kill target-antigen-positive human cancer cells in vitro within co-culture assays. The NV TRAC-CAR T cells successfully cause tumor regression in vivo within human xenograft cancer models in mice at comparable efficiency to state-of-the-art, viral CAR T cells. NV-TRAC-CAR T cells can be manufactured in a xeno-free manner and have high potential to simplify and advance CAR T cell manufacturing by elimination of viral vectors.

In an aspect, an ex vivo method of site-specifically inserting a synthetic DNA sequence, e.g., a transgene containing a chimeric antigen receptor (CAR) gene, into a T cell genome comprises introducing into a population of unmodified T cells a Cas9 ribonucleoprotein (RNP) and a non-viral double-stranded homology-directed repair (HDR) template, to provide genome-edited T cells. The Cas9 RNP comprises a Cas9 protein and a guide RNA that directs double stranded DNA cleavage of a cleavage site in a T-cell expressed gene. The non-viral double-stranded HDR template comprises a synthetic DNA sequence flanked by homology arms that are complementary to sequences on both sides of the cleavage site in the T cell expressed gene. The synthetic DNA sequence is specifically integrated into the cleavage site of the T cell expressed gene by the Cas9 ribonucleoprotein in the genome-edited T cells. After integration, the method includes culturing the genome-edited T cells in xeno-free medium to provide a cultured population of genome-edited T cells having the synthetic DNA sequence specifically integrated in the T-cell expressed gene locus. In the cultured population of genome-edited T cells, an endogenous promoter of the T cell expressed gene drives expression of the synthetic DNA sequence, or the synthetic DNA sequence includes a promoter that drives expression of the synthetic DNA sequence.

In the methods described herein, a synthetic DNA sequence is site-specifically inserted into the genome of a T cell, specifically into a T cell expressed gene. As used herein, a synthetic DNA sequence is a DNA sequence that is not native to the genome of the T cell to be modified. An exemplary aspect of a synthetic DNA sequence is a “chimeric antigen receptor (CAR)”. CAR refers to a recombinant fusion protein that has an antigen-specific extracellular domain coupled to an intracellular domain that directs the cell to perform a specialized function upon binding of an antigen to the extracellular domain. In an aspect, a CAR comprises an antigen-specific extracellular domain (e.g., a single chain variable fragment [scFV] that can bind a surface-expressed antigen of a malignancy) coupled to an intracellular domain (e.g., CD28, ICOS, CD27, 4-1BB, OX40, CD40L, or CD3-ζ) by a transmembrane domain (e.g., derived from a CD4, CD8α, CD28, IgG or CD3-ζ transmembrane domain).

In an aspect, the length of the homology arms influences the efficiency of synthetic DNA sequence integration. In an aspect, the homology arms are 400 to 1000 base pairs, specifically 450 to 750 base pairs long.

The antigen-specific extracellular domain of a CAR recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 μM, specifically about 0.1 pM to about 1 μM, more specifically about 0.1 pM to about 100 nM. Methods for determining the affinity of interaction are known in the art. An antigen-specific extracellular domain suitable for use in a CAR may be any antigen-binding polypeptide, one or more scFv, or another antibody based recognition domain (cAb VHH (camelid antibody variable domains) or humanized versions thereof, IgNAR VH (shark antibody variable domains) and humanized versions thereof, sdAb VH (single domain antibody variable domains) and “camelized” antibody variable domains are suitable for use. In some instances, T cell receptor (TCR) based recognition domains such as single chain TCR may be used as well as ligands for cytokine receptors.

The present disclosure provides chimeric antigen receptors (CARs) that bind to an antigen of interest. The CAR can bind to a tumor antigen or a pathogen antigen.

In certain embodiments, the CAR binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB.

In certain embodiments, the CAR binds to a pathogen antigen, e.g., for use in treating and/or preventing a pathogen infection or other infectious disease, for example, in an immunocompromised subject. Non-limiting examples of pathogen include viruses, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcusfaecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

The intracellular domain transmits the T cell activation signal. The intracellular domain can increase CAR T cell cytokine production and facilitate T cell replication. The intracellular domain reduces CAR T cell exhaustion, increases T cell antitumor activity, and enhances survival of CAR T cells in patients. Exemplary intracellular domains, also call co-stimulatory domains, include CD28, ICOS, CD27, 4-1BB, OX40, CD40L, and CD3-ζ.

Typically, the antigen-specific extracellular domain is linked to the intracellular domain of the CAR by a transmembrane domain, e.g., derived from a CD4, CD8α, CD28, IgG or CD3-ζ transmembrane domain. The transmembrane domain traverses the cell membrane, anchors the CAR to the T cell surface, and connects the extracellular domain to the intracellular signaling domain, thus impacting expression of the CAR on the T cell surface. CARs may also further comprise one or more costimulatory domain and/or one or more spacer. A costimulatory domain is derived from the intracellular signaling domains of costimulatory proteins that enhance cytokine production, proliferation, cytotoxicity, and/or persistence in vivo. A spacer or hinge connects (i) the antigen-specific extracellular domain to the transmembrane domain, (ii) the transmembrane domain to a costimulatory domain, (Hi) a costimulatory domain to the intracellular domain, and/or (iv) the transmembrane domain to the intracellular domain. For example, inclusion of a spacer domain (e.g. IgG1, IgG2, IgG4, CD28, CD8) between the antigen-specific extracellular domain and the transmembrane domain may affect flexibility of the antigen-binding domain and thereby CAR function. Suitable transmembrane domains, costimulatory domains, and spacers are known in the art.

In an aspect, synthetic DNA sequences within the HDRT could incorporate synthetic receptors, cytokine signaling, and short hairpin (sh)RNA. One example is to make use of natural ligand—receptor pairs (e.g., modified interleukin (IL)-13 sequences) and natural ligand-binding domains of receptors (e.g., NKG2D and CD27) to target receptors to disease. Another example is incorporate sequences that encode cytokine receptor signaling important for T cell maintenance and expansion (e.g., IL-2 receptor beta chain (IL-2Rb) and a STAT3-binding motif). In addition, sh(RNA) could also be expressed from the synthetic DNA sequence that helps provide control over the edited T cell behavior.

In an aspect, the synthetic DNA sequence comprises a coding sequence for a fluorescent protein such as mCherry, mKate, GFP, BFP, RFP, CFP, YFP, mCyan, mOrange, tdTomato, mBanana, mPlum, mRaspberry, mStrawberry, and mTangerine.

In order to insert the synthetic DNA sequence into the genome of the unmodified T cells, a Cas9 RNP and a non-viral double-stranded HDR template including the synthetic DNA sequence are introduced into the unmodified T cells to provide genome-edited T cells.

As used herein, “introducing” means refers to the translocation of the Cas9 ribonucleoprotein and a non-viral double-stranded HDR template from outside a cell to inside the cell, such as inside the nucleus of the cell. Introducing can include transfection, electroporation, contact with nanowires or nanotubes, receptor mediated internalization, translocation via cell penetrating peptides, liposome mediated translocation, and the like.

Unmodified T cells include autologous T cells that are collected from a patient, such as a cancer patient, by peripheral blood draw or leukapheresis. Unmodified T cells can also include T cells from allogeneic healthy donors or induced pluripotent stem cells which can be used to produce universal T cells for administration to a patient. T cells are generally modified ex vivo, that is outside of the patient, and then the modified T cells such as CAR T cells are returned to the patient, such as by intravenous infusion, subcutaneous, intratumoral, intraperitoneal or intracerebral injection.

Genome editing of the T cells as described herein uses a CRISPR system, or Cas9 ribonucleoprotein. CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. Guide RNA base pairs with complementary sequences in target DNA. Cas9 nuclease activity then generates a double-stranded break in the target DNA.

CRISPR/Cas9 is a ribonucleoprotein (RNP) complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or an sgRNA single guide RNA. The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer sequence refers to the nucleotide sequence of a guide RNA that binds to a target genomic DNA sequence and directs Cas9 nuclease activity to a target DNA locus in the genome of the T cell such the TRAC gene, a T cell receptor beta subunit constant gene (TRBC), AAVS1 (i.e., PPP1R12C), TET2, FAS, BID, CTLA4, PDCD1, CBLB, PTPN6, CIITA and B2M genes. In some embodiments, the guide RNA protospacer sequence is complementary to the target DNA sequence. “Complementary” or “complementarity” refers to specific base pairing between nucleotides or nucleic acids. Base pairing between a guide RNA and a target region in exon 1 of the TRAC gene can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT; the PAM motif of S. thermophilus Cas9 is NNAGAAW; and the PAM motif of T denticola Cas9 is NAAAAC.

A “Cas9” polypeptide is a polypeptide that functions as a nuclease when complexed to a guide RNA, e.g., an sgRNA or modified sgRNA. That is, Cas9 is an RNA-mediated nuclease. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

Exemplary Cas9 polypeptides include Cas9 polypeptide derived from Streptococcus pyogenes, e.g., a polypeptide having the sequence of the Swiss-Prot accession Q99ZW2 (SEQ ID NO: 5); Cas9 polypeptide derived from Streptococcus thermophilus, e.g., a polypeptide having the sequence of the Swiss-Prot accession G3ECR1 (SEQ ID NO: 6); a Cas9 polypeptide derived from a bacterial species within the genus Streptococcus; a Cas9 polypeptide derived from a bacterial species in the genus Neisseria (e.g., GenBank accession number YP_003082577; WP_015815286.1 (SEQ ID NO: 7)); a Cas9 polypeptide derived from a bacterial species within the genus Treponema (e.g., GenBank accession number EMB41078 (SEQ ID NO: 8)); and a polypeptide with Cas9 activity derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

As used herein, the term editing refers to a change in the sequence of the genome at a targeted genomic location. Editing can include inducing either a double stranded break or a pair of single stranded breaks in the genome, such as in a T cell expressed gene. Editing can also include inserting a synthetic DNA sequence into the genome of the T cell at the site of the break(s).

As used herein, a Cas9 RNP that targets a T cell expressed gene comprises a Cas9 protein and a guide RNA that directs double stranded cleavage of the T cell expressed gene. The guide RNA thus includes a crRNA comprising a single-stranded protospacer sequence and a first complementary strand of a binding region for the Cas9 polypeptide, and a tracrRNA comprising a second complementary strand of the binding region for the Cas9 polypeptide, wherein the crRNA and the tracrRNA hybridize through the first and second complementary strands of the binding region for the Cas9 polypeptide. The single-stranded protospacer region of the guide RNA hybridizes to a sequence in the T cell expressed gene, directing cleavage of the T-cell expressed gene to a specific locus of the T cell expressed gene.

Exemplary T cell expressed genes which can be cleaved by the methods described herein include the AAVS1 (i.e., PPP1R12C), TET2, FAS, BID, CTLA4, PDCD1, CBLB, PTPN6, CIITA, B2M, TRAC and TRBC genes, specifically TRAC. The T cell expressed gene-targeting Cas9 ribonucleoprotein results in a reduction or elimination of expression of functional TRAC gene product (e.g., knockout of expression of functional TRAC gene product).

In an aspect, the T cell expressed gene is TRAC and wherein the guide RNA targets the 5′ end of the first exon of TRAC. An exemplary guide RNA useful to target exon 1 of TRAC comprises SEQ ID NO: 9.

In addition to the Cas9 RNP, a non-viral double-stranded HDR template comprising the synthetic DNA sequence is introduced into the T cells. In prior art methods, viral vectors such as adeno-associated virus vectors have been used to provide the synthetic DNA template. Even when combined with Cas9 RNP gene editing, the use of AAV vectors (a) are expensive; (b) could integrate viral genomes into the human genome; (c) trigger an immune response within the patient to viral components; (d) may result in highly variable transgene expression; and (d) take extended periods of time (e.g., months to years) to manufacture.

In an aspect, the non-viral double-stranded HDR template comprises the synthetic DNA sequence flanked by homology arms for insertion of the synthetic DNA sequence into the T cell expressed gene by the Cas9 RNP. The homology arms have 50 to 3000 nucleotides in length and are complementary to sequences on either side of the cut site in the T cell expressed gene to facilitate incorporation of the synthetic DNA sequence into the genome of the T cell. Small sequence variations (<100 bases) from complementary sequences could be included to enable barcoding or tracking of various cell types. For example, when the T cell expressed gene comprises exon 1 of TRAC, the homology arms can comprise:

In an aspect, the non-viral double-stranded HDR template sequentially comprises a left homology arm—a splice acceptor site—a self-cleaving peptide sequence (e.g., a T2A coding sequence)—a CAR gene—a polyA terminator—a right homology arm.

The splice acceptor site assists in the splicing of the synthetic DNA sequence into the transcript generated from the native T cell expressed gene.

The self-cleaving peptide sequence, e.g., T2A, assists in the separation or cleavage of the translated peptide of the protein product encoded by the synthetic DNA sequence from the protein product of the native T cell expressed gene. Exemplary self-cleaving peptides sequences include viral 2A peptides such as the a porcine teschovirus-1 (P2A) peptide, a Thosea asigna virus (T2A) peptide, an equine rhinitis A virus (E2A) peptide, or a foot-and-mouth disease virus (F2A) peptide.

The polyA terminator, e.g., a bovine growth hormone polyA. The polyA terminator is a sequence-based element that defines the end of a transcriptional unit within the synthetic DNA sequence and initiate the process of releasing the newly synthesized RNA from the transcription machinery.

In an aspect, the non-viral double-stranded HDR template is produced by amplifying a sequence from a bacterial plasmid, e.g., SEQ ID NO: 1. Amplification can be done using a Q5® Hot Start Polymerase (NEB).

Also included herein is the plasmid of SEQ ID NO: 1. In an aspect, the double-stranded HDR template has an OD260/OD280 of 1.8 to 2.1, and/or an OD260/OD230 of 2.0 to 2.3.

In an aspect, the double-stranded HDR template has a concentration of 2000 to 10000 ng/μl.

After introducing the Cas9 RNP and a non-viral double-stranded HDR template into the unmodified T cells, a population of genome-edited T cells is produced.

In an aspect, the genome-edited T cells are deficient in expression of the T-cell expressed gene product, while expressing a gene product of the synthetic DNA sequence. The endogenous promoter of the T-cell expressed gene can drive expression of a gene product within the synthetic DNA sequence.

The genome-edited T cells are then cultured in in xeno-free medium to provide a cultured population of T cells having the synthetic DNA sequence specifically integrated in the T-cell expressed gene locus. The term “xeno” comes from the Greek “xenos” meaning strange. Zeno-free (or xenogeneic-free) therefore means free from “strange” components, or components from a “strange” species (strange being relative to the native species you're working with). In terms of cell culture, this would mean human cell lines can be cultured using human-derived components (like human serum), and it is considered xeno-free, since there is no difference between species.

As used herein culturing the genome-edited T cells in xeno-free medium can include recovery from integration of the synthetic DNA sequence and/or expansion of the edited T cell population.

In an aspect, after culturing, the modified T cells can aggregate to form a cluster of cells. Cells which exhibit a higher degree of aggregation typically recover at higher rates than cells that do not aggregate. The aggregation could help cell-cell interaction through paracrine or juxtacrine signaling that assists in recovery. In an aspect, the method further comprises imaging the population of CAR T cells and determining the degree of aggregation of the CAR T cells, and optionally selecting a population of aggregated CAR T cells.

In an aspect, culturing is done in round bottom culture wells at 20% of standard culture volume for the wells. It was unexpectedly found that by using round bottom culture wells rather than flat, for example, improved recovery was observed.

In the methods described herein, more than 4, 5, 6, 7, 8, 9 or 10% of the population of unmodified T cells has the synthetic target gene inserted into their genomes.

In the cultured population of genome-edited T cells, in the cultured population of genome-edited T cells, an endogenous promoter of the T cell expressed gene drives expression of the synthetic DNA sequence, or the synthetic DNA sequence can include a promoter that drives expression of the synthetic DNA sequence. Exemplary promoters include CAGGS and EF1alpha.

In an aspect, the CAR T cells produced by the methods described herein have activity against an antigen on a solid tumor in vitro or in vivo.

In an aspect, described herein is a non-viral produced CAR T cell with a genome having a CAR sequence specifically integrated into a T cell expressed gene, wherein the T cell is enriched for the CD62L and/or CD45RA markers indicative of naïve and stem cell memory phenotypes compared to viral-produced control CAR T cells. Also included is a non-viral produced CAR T cell with a genome having a CAR sequence specifically integrated into a T cell expressed gene, wherein the T cell has reduced expression of TIM3 and/or LAG3 markers of T cell exhaustion compared to viral-produced control CAR T cells. The CD62L and/or CD45RA markers can be enriched more than 2-fold compared to the CD62L and/or CD45RA markers compared to viral-produced control CAR T cells. In another aspect, the TIM3 and/or LAG3 markers are reduced more than 2-fold compared to the TIM3 and/or LAG3 markers compared to viral-produced control CAR T cells.

Also include herein are method of treating a subject comprising administering any of the foregoing genome-edited T cells comprising the synthetic DNA sequence to a subject in need of T cell therapy, such as CART cell therapy. CAR T cell therapy, for example, has been approved to treat hematologic malignancies like acute lymphoblastic leukemia, non-Hodgkin large B-cell lymphomas, and have been used to treat chronic lymphocytic leukemia and multiple myeloma. Herein evidence is presented a method that produces CAR T cells that should not only be active against hematologic malignancies, but could be used to produce CAR T cells with activity against solid tumors. CAR T cells are typically administered by intravenous infusion but could also be administered by subcutaneous, intratumoral, intraperitoneal or intracerebral injection.

The invention is further illustrated by the following non-limiting examples.

### EXAMPLES

### Materials and Methods

Data Reporting. For in vivo experiments, established tumor burden was verified by IVIS luciferase imaging prior to infusion. Mice were arranged according to tumor burden and distributed evenly across conditions. The experiments were not randomized and the investigators were not blinded during experiments and outcome assessment.

Antibodies. Antibodies used in this study for flow cytometry and fluorescence activated cell sorting are listed in Table 1.

TABLE 1
Antibodies used in flow cytometry and cell experiments

| Antigen | Clone | Fluorophore |
|---|---|---|
| CAR | anti-id | APC |
| | 1A7 | |
| CCR7 | G043H7 | Brilliant |
| | | Violet ™ 711 |
| CCR7 | G043H7 | Brilliant |
| | | Violet ™ 650 |
| CD19 | HIB19 | APC-Fire ™ |
| | | 750 |
| CD3 | OKT3 | AlexaFluor ® |
| | | 488 |
| CD3 | OKT3 | Brilliant |
| | | Violet ™ 785 |
| CD3 | OKT3 | PE-Dazzle ™ |
| | | 594 |
| CD3 | OKT3 | AlexaFluor ® |
| | | 488 |
| CD4 | OKT4 | Brilliant |
| | | Violet ™ 711 |
| CD4 | OKT4 | PE-Cyanine5 |
| CD45RA | HI100 | PE-Cyanine7 |
| CD62L | DREG56 | Brilliant |
| | | Violet ™ 605 |
| CD62L | DREG56 | PE |
| CD69 | FN50 | PE-Dazzle ™ |
| | | 594 |
| CD8 | SK1 | PerCP- |
| | | eFluor710 |
| CD95 | DX2 | AlexaFluor ® |
| | | 700 |
| GD2 | 14G2a | APC |
| Human CD45 | HI30 | Pacific Blue |
| IgG2a | RMG2a-62 | APC |
| LAG3 | 3DS223H | PE |
| Mouse CD45.1 | A20 | PE-Cyanine7 |
| PD1 | EH12.2H7 | AlexaFluor ® |
| | | 488 |
| TCR α/β | IP26 | BV421 |
| TCR α/β | IP26 | AlexaFluor ® |
| | | 488 |
| TIM3 | F38-2E2 | Brilliant |
| | | Violet ™ 510 |
| Mouse Lyt2 | 53-6.7 | AlexaFluor ® |
| | | 488 |
| GhostRed ™780 | Viability | — |

Guide RNAs. All guide RNAs used in this study are listed in Table 2.

TABLE 2
gRNA sequences

| gRNA | Sequence | SEQ ID NO: |
|---|---|---|
| TRAC | 5′ CAGGGTTCTGGATATCTGT 3′ | 9 |

The full sequence of the cr RNA is 5′ CAGGGTTCTGGATATCTGTGTTTTAGAGCTATGCT3′ (SEQ ID NO: 10). The tracr portion of the guide RNA is a proprietary 67mer tracr RNA available from IDT.

Primers. All primers used in this study are listed in Table 3.

TABLE 3

| | | SEQ |
|---|---|---|
| Oligo | Sequence | ID NO: |
| TRAC Donor | CCTTTTTCCCATGCCTGCCTTT | 11 |
| FWD primer | | |
| TRAC Donor | TAAGGCCGAGACCACCAATCAG | 12 |
| REV primer | | |
| TRAC sequencing | ACACTCTTTCCCTACACGACGCTCTT | 13 |
| FWD primer | CCGATCT | |
| TRAC sequencing | GTGACTGGAGTTCAGACGTGTGCTCT | 14 |
| REV primer | TCCGATCT | |
| TRAC genomic | ATCTTGTGCGCATGTGAGGGGC | 15 |
| integration | | |
| FWD primer | | |
| TRAC genomic | GCAAGCCAGGACTCCACCAACC | 16 |
| integration | | |
| REV primer | | |

Cell lines. CHLA-20 human neuroblastoma were a gift from Dr. Maria Otto. These cells were maintained in Dulbecco's Modified Eagle Medium high glucose (Gibco) supplemented with 10% Fetal Bovine Serum (Gibco) and 1% Penicillin-Streptomycin. AkaLuc-GFP CHLA-20 cells were a gift from the J. Thomson lab (UW-Madison). Phoenix™ cells (ATCC) for viral preparation were maintained in DMEM (high glucose) supplemented with 10% Fetal Bovine Serum (Gibco), and selected using 1 μg/mL diphtheria toxin and 300 μg/mL hygromycin prior to use. Selection for transgene positive cells was confirmed by flow cytometry for Lyt2 expression (Biolegend) (>70%+). 3T3 cells were maintained in Dulbecco's Modified Eagle Medium (Gibco) supplemented with 10% Fetal Bovine Serum (Gibco) and 1% Penicillin-Streptomycin (Gibco). Cell authentication was performed using short tandem repeat analysis (Idexx BioAnalytics, Westbrook, Me.) and per ATCC guidelines using morphology, growth curves, and Mycoplasma testing within 6 months of use using the e-Myco mycoplasma PCR detection kit (iNtRON Biotechnology Inc, Boca Raton, Fla.). Cell lines were maintained in culture at 37° C. in 5% CO₂, and used after 3-5 passages in culture after thawing.

Plasmid constructs. NV-AAVS1-CAR: An NV-AAVS1-CAR donor plasmid (SEQ ID NO: 1) was designed using a pAAV-CAGGS-GFP backbone (Addgene) and a 2 kb CAR gBlock (IDT), which was inserted into the backbone using restriction cloning. NV-TRAC-CAR: A 2 kb region surrounding the TRAC locus was amplified by PCR from human genomic DNA and cloned into a pCR blunt II TOPO™ backbone (Thermo Fisher Scientific). The CAR transgene was then cloned into the TOPO™ TRAC vector using Gibson Assembly (NEB). Plasmid sequence was verified by Sanger sequencing. TRAC-H2B-mCherry, NV-TRAC-41bb-CAR: These constructs were ordered as synthesized genes in a pUC57 vector (GenScript). All plasmids were grown in NEB®5-alpha competent E. coli (NEB) and purified using the PureYield™ MidiPrep system (Promega).

Double-stranded DNA HDRT production. Plasmid constructs were used as PCR templates for NV (nonviral) products. In brief, NV-CAR and NV-mCh plasmids were MidiPrepped using the PureYield MidiPrep system (Promega). PCR amplicons were generated from plasmid templates using Q5® Hot Start Polymerase (NEB), and pooled into 100 μl reactions for Solid Phase Reversible Immobilization (SPRI) cleanup (1×) using AMPure® XP beads according to the manufacturer's instructions (Beckman Coulter). Each 100 μl starting product was eluted into 5 μl of water. Bead incubation and separation times were increased to 5 minutes, and elution time was increased to 15 minutes at 37° C. to improve yield. PCR products from round 1 cleanup were pooled and subjected to a second round of SPRI cleanup (1×) to increase total concentration; round 2 elution volume was 20% of round 1 input volume. Template concentration and purity was quantified using NanoDrop™ 2000 and Qubit™ dsDNA BR Assays (Thermo Fisher Scientific), and templates were diluted in water to an exact concentration of 2 μg/μl.

SpyCas9 RNP preparation. RNPs were produced by complexing a two-component gRNA to SpyCas9. In brief, tracrRNA and crRNA were ordered from IDT, suspended in nuclease-free duplex buffer at 100 μM, and stored in single-use aliquots at −80° C. tracrRNA and crRNA were thawed, and 1 μl of each component was mixed 1:1 by volume and annealed by incubation at 37° C. for 30 minutes to form a 50 μM gRNA solution in individual aliquots for each electroporation replicate. Recombinant sNLS-SpCas9-sNLS Cas9 (Aldevron, 10 mg/ml, total 0.8 μl) was added to the complexed gRNA at a 1:1 molar ratio and incubated for 15 minutes at 37° C. to form an RNP. Individual aliquots of RNPs were incubated for at least 30 seconds at room temperature with HDR templates for each sample prior to electroporation.

Isolation of human primary T cells. This study was approved by the Institutional Review Board of the University of Wisconsin-Madison (#2018-0103), and informed consent was obtained from all donors. Peripheral blood was drawn from healthy donors into sterile syringes containing heparin, then transferred to sterile 50 mL conical tubes. Primary human T cells were isolated using negative selection per the manufacturer's instructions (RosetteSep™ Human T Cell Enrichment Cocktail, STEMCELL Technologies). T cells were counted using a Countess™ II FL Automated Cell Counter with 0.4% Trypan Blue viability stain (Thermo Fisher). T cells were cultured at a density of 1 million cells/mL in ImmunoCult™-XF T cell Expansion Medium (STEMCELL) supplemented with 200 U/mL IL-2 (Peprotech) and stimulated with ImmunoCult™ Human CD3/CD28/CD2 T cell Activator (STEMCELL) immediately after isolation, per the manufacturer's instructions.

T cell culture. Bulk T cells were cultured in ImmunoCult™-XF T cell Expansion Medium at an approximate density of 1 million cells/mL. In brief, T cells were stimulated with ImmunoCult™ Human CD3/CD28/CD2 T cell Activator (STEMCELL) for 2 days prior to electroporation. On day 3, (24 hours post-electroporation), NV T and NV-mCh T cells were transferred to 1 mL of fresh culture medium (with 500 U/mL IL-2, without activator) and allowed to expand. T cells were passaged, counted, and adjusted to 1 million/mL in fresh medium +IL-2 on days 5 and 7 after isolation. RV-CAR T cells were spinoculated with RV-CAR construct on day 3 and passaged on day 5 with the NV-CAR and NV-mCh T cells. Prior to electroporation or spinoculation, the medium was supplemented with 200 U/mL IL-2; post gene editing, medium was supplemented with 500 U/mL IL-2 (Peprotech).

T cell electroporation. RNPs and HDR templates were electroporated 2 days after T cell isolation and stimulation. During crRNA and tracrRNA incubation, T cells were centrifuged for 3 minutes at 200 g and counted using a Countess™ II FL Automated Cell Counter with 0.4% Trypan Blue viability stain (Thermo Fisher). 1 million cells per replicate were aliquoted into 1.5 mL tubes. During RNP complexation step (see RNP production), T cell aliquots were centrifuged for 10 min at 90 g. During the spin step, 2 μl HDR template (total 4 μg) per condition were aliquoted to PCR tubes, followed by RNPs (2.8 μl per well; pipette should be set to a higher volume to ensure complete expulsion of the highly viscous solution). Templates and RNPs were incubated at room temperature for at least 30 seconds. After cell centrifugation, supernatants were aspirated, and cells were resuspended in 20 μl P3 buffer, then transferred to PCR tubes containing RNP. 24 μl total volume per sample was transferred directly into wells of the 16 well Nucleocuvette™ strips. Typically, no more than 8 reactions were completed at a time to minimize the amount of time T cells spend in P3 buffer. T cells were electroporated with a Lonza 4D Nucleofector™ with X Unit using pulse code EH115. Immediately after nucleofection, 80 μl of pre-warmed media with 500 U/mL IL-2 and 25 μl/mL ImmunoCult™ CD3/CD28/CD2 activator was added to each cuvette well. Cuvettes rested at 37° C. in the cell culture incubator for 15 minutes. After 15 minutes, cells were moved to 200 μl total volume of media+IL-2+activator (see Primary T cell culture above) in a round bottom 96 well plate.

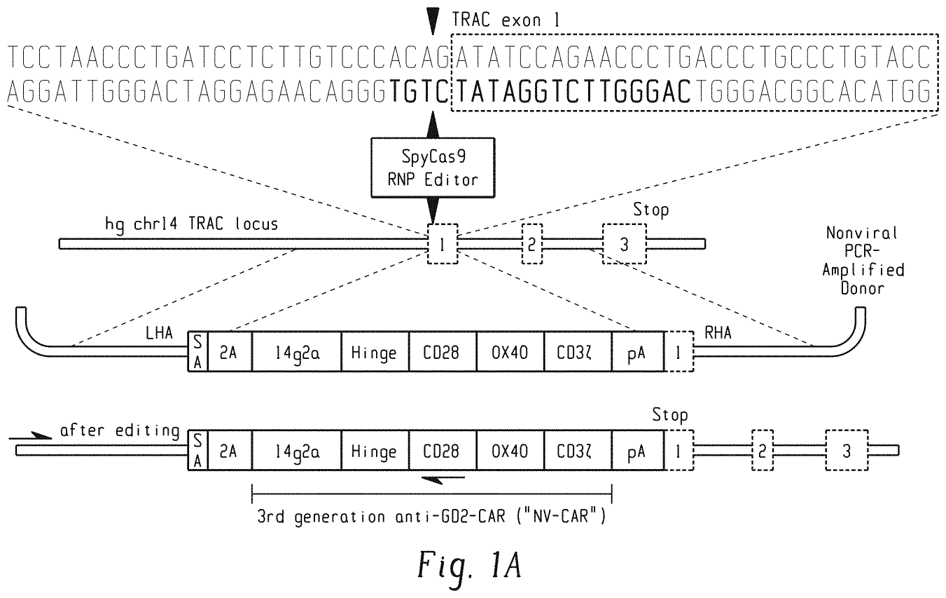

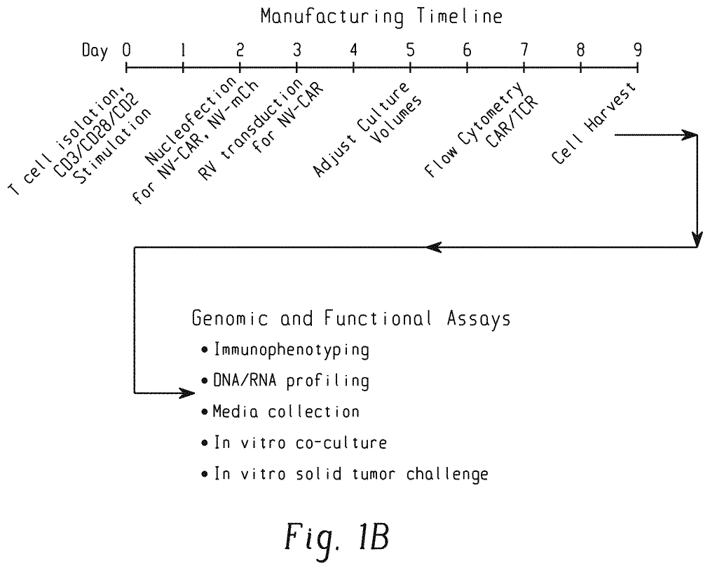

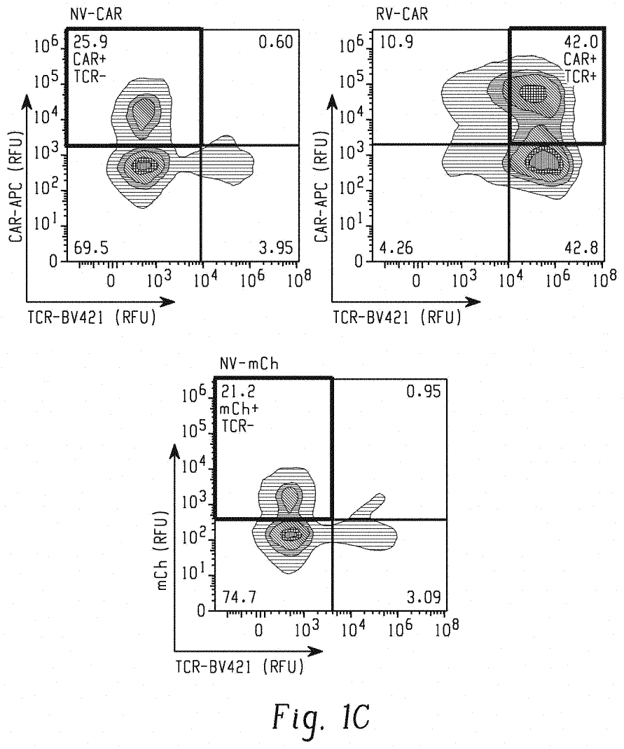

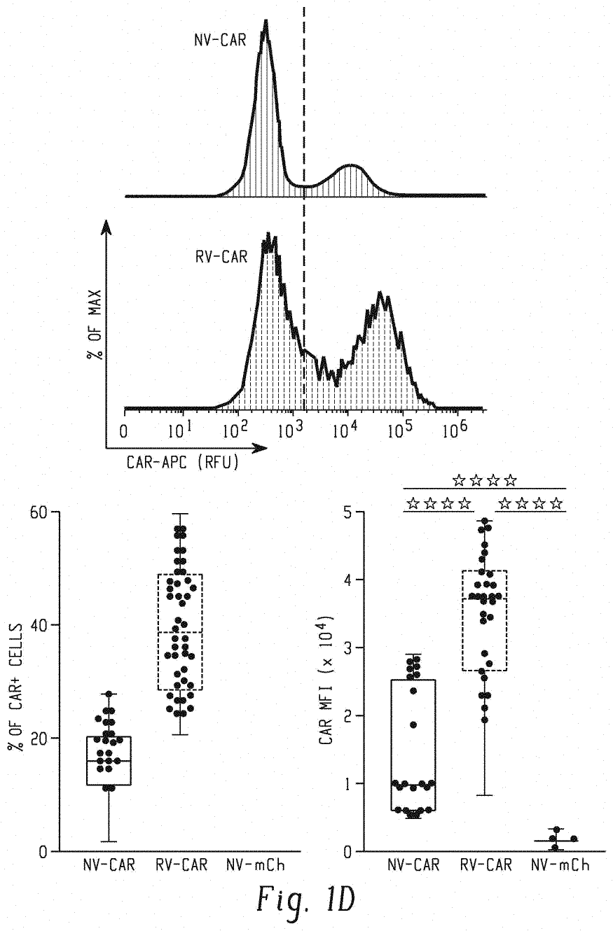

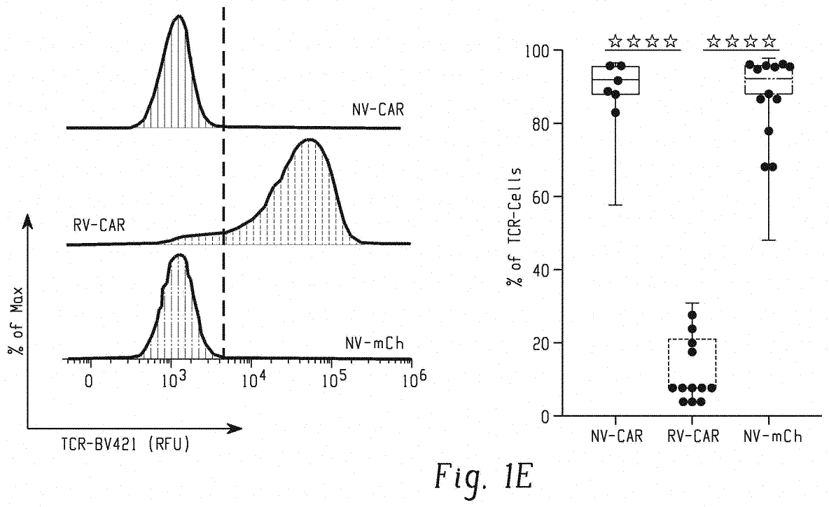

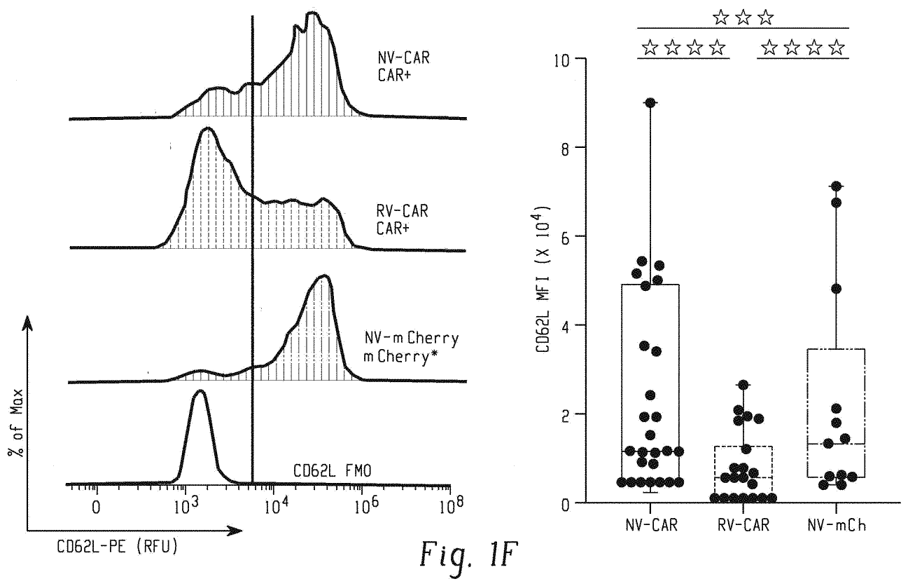

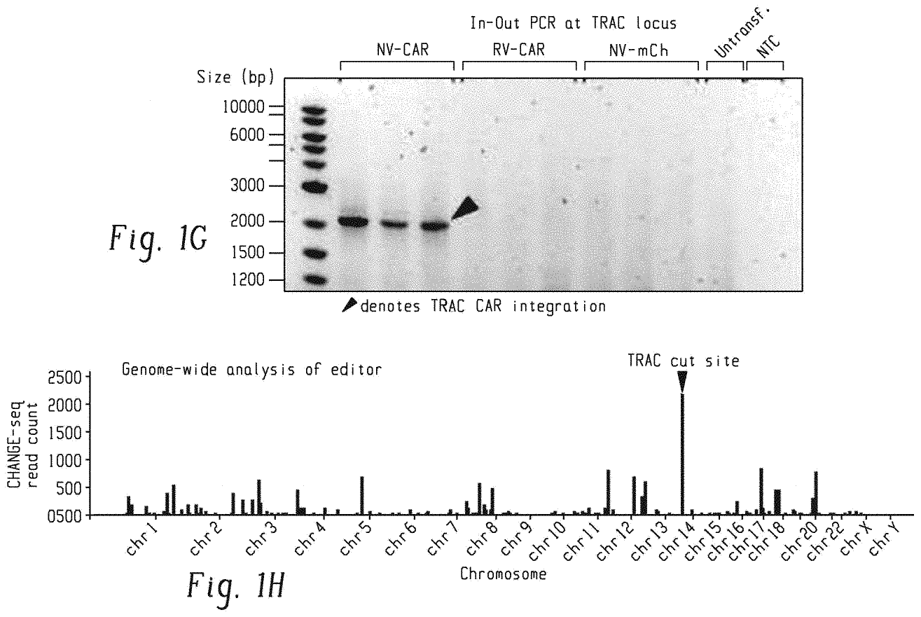

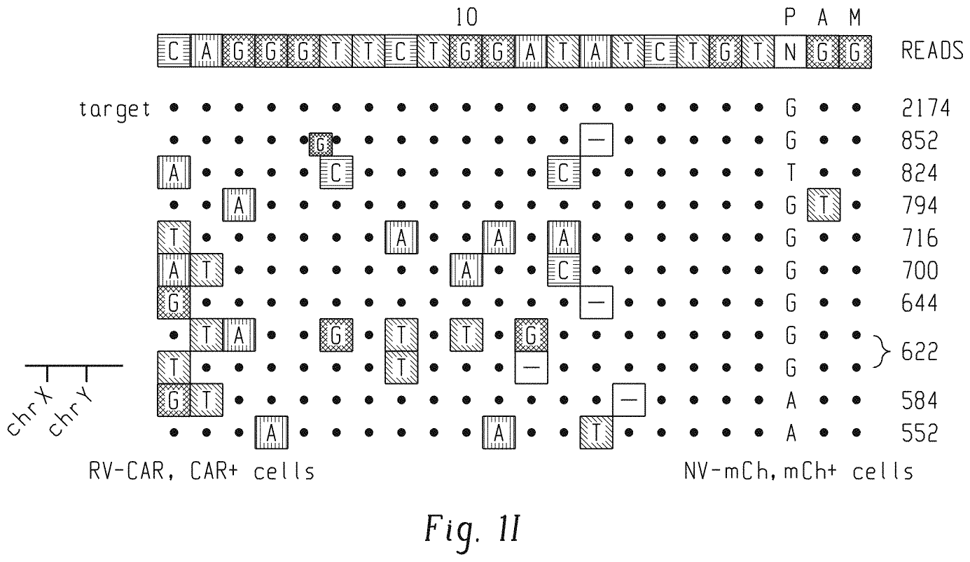

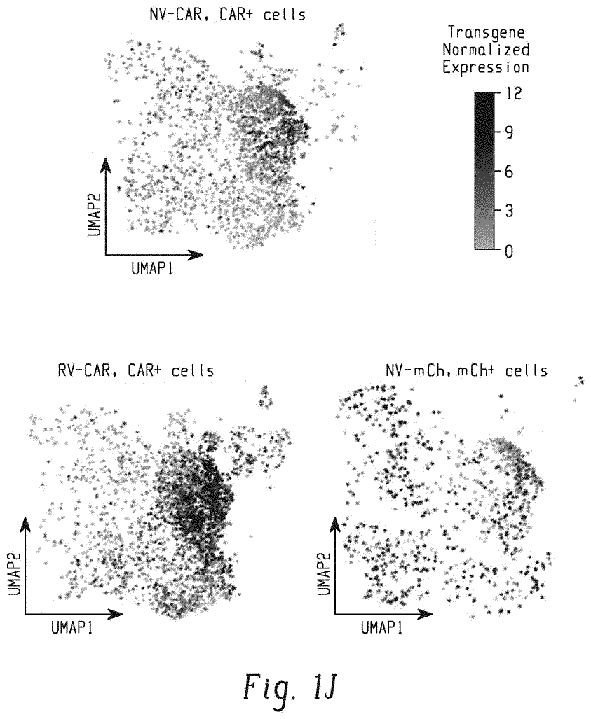

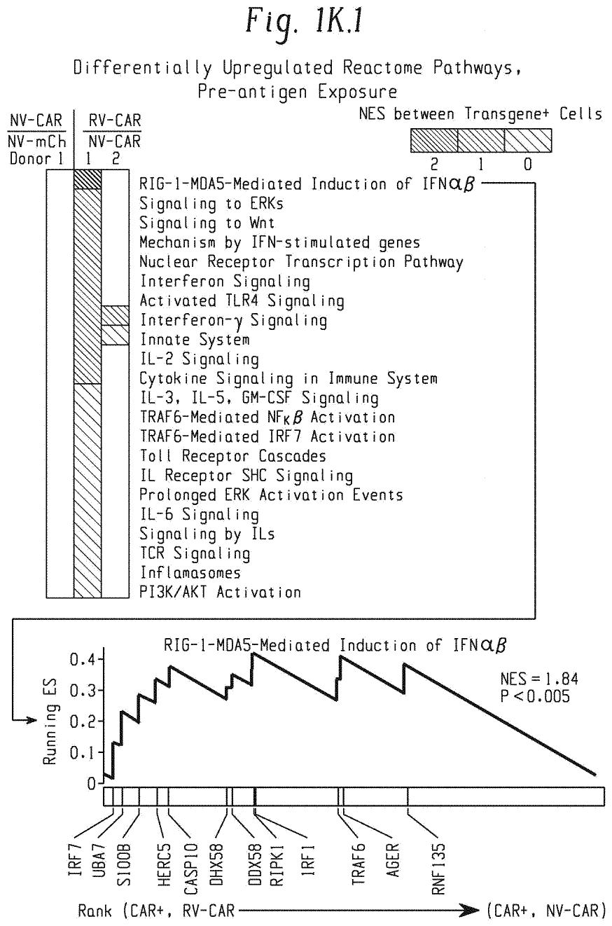

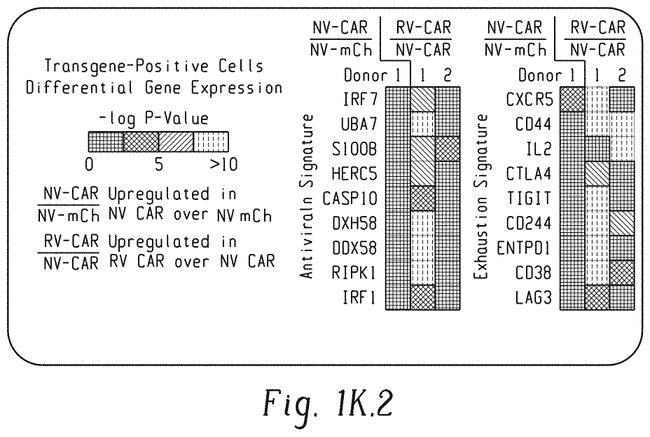

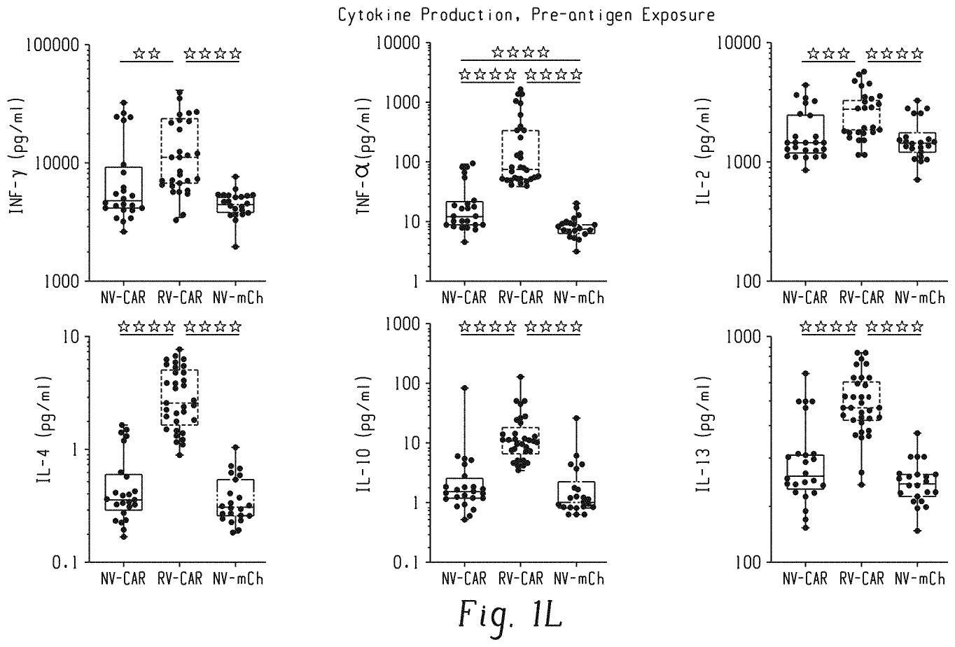

Retrovirus production. CAR retrovirus was manufactured using Phoenix™(ATCC). In brief, pSFG.iCasp9.2A.14G2A-CD28-OX40-CD3z plasmid (was MidiPrepped using the PureYield™ MidiPrep system (Promega). One day prior to transfection, selected Phoenix™ cells were plated on 0.01% Poly-L-Lysine coated 15 cm dishes at a density of 76,000 cells/cm², or ˜65% confluency. On transfection day, media was replaced 1 hour prior to transfection of 10 μg pSFG.iCasp9.2A.14G2A-CD28-OX40-CD3z plasmid/plate using iMFectin according to the manufacturer's instructions (GenDEPOT). Media was replaced 18-24 hours later with 10 mL of 50 mM HEPES buffered DMEM +10% FBS (Gibco). 48 hours later, media was collected, stored at 4° C., and replaced. A second aliquot of media was collected 24 hours later. A second aliquot of media was collected 24 hours later; media aliquots were pooled and centrifuged for 10 min at 2000 g to pellet contaminating cells, and supernatants were transferred to a clean conical tube. ⅓ volume Retro-X™ concentrator (Takara/Clonetech) was added, and supernatants were refrigerated at 4° C. for 12-18 hours, and then concentrated according to the manufacturer's instructions. Viruses were tested on 3T3 cells prior to use. Yields from one 15 cm dish were used for 5 replicate conditions, where each replicate consisted of a well with 160,000 T cells per transduction. Viruses were either used immediately for T cell spinoculation, or stored at −80° C. in single use aliquots. Retroviral transduction. T cells for RV infection were cultured similarly to NV T and NV-mCh T cells, with two exceptions: 1) T cells were passaged and resuspended without Immunocult™ CD2/CD28/CD3 activator on day 2 post-isolation, then spinoculated on Day 3. RV-CAR T cells were returned to the regular passaging schedule on day 5 post-isolation. (See FIG. 1b). Prior to spinoculation, non-treated cell culture 24 well plates were coated with Retronectin® according to the manufacturer's instructions (Takara/Clontech). On day 3 post-isolation, T cells were centrifuged at 200 g for 3 minutes, counted, and resuspended to a concentration of 200,000 cells/mL, then stored in the incubator until plates were prepared. Virus was added to Retronectin®-coated plates in a volume of 400 μl virus+ImmunoCult™ medium and centrifuged at 2000 g for 2 hours at 32C. 160,000 T cells in 800 μl were added to each well and spinoculated at 2000 g for 60 minutes at 32° C., without brakes. T cells were then transferred to the incubator and left undisturbed for two days.

Flow cytometry and fluorescence activated cell sorting. CAR was detected using 1A7 anti-14G2a idiotype antibody (gift from Paul Sondel) conjugated to APC with the Lightning-Link® APC Antibody Labeling kit (Novus Biologicals). T cells were stained in BD Brilliant™ Stain Buffer (BD Biosciences). For panels including TRAC and CD3, cells were permeabilized and fixed using the BD Cytofix/Cytoperm™ Plus kit according to the manufacturer's instructions. Flow cytometry was performed on an Attune™ N×T Flow cytometer, and fluorescence-activated cell sorting was performed on a BD FACS Aria™. All antibodies used in this study are described in Table 1. T cells from Donors 1 and 2 were stained and analyzed on day 9 of manufacture using fresh cells. For donors 3 and 4, only TCR, CAR, and CD62L were measured on day 9 of manufacture. The change in protocol was made due to equipment restrictions related to institutional COVID-19 biosafety precautions, and CD62L was selected for analysis due to the known effects of cryopreservation on expression levels.

“In-Out PCR”. Genomic DNA was extracted from 100,000 cells per condition using DNA QuickExtract™ (Lucigen), and incubated at 65° C. for 15 min, 68° C. for 15 min, and 98° C. for 10 min. Genomic integration of the CAR was confirmed by In-out PCR using a forward primer upstream of the TRAC left homology arm, and a reverse primer binding within the CAR sequence. Primer sequences are listed in Table 3. PCR was performed according to the manufacturer's instructions using Q50® Hot Start Polymerase (NEB) using the following program: 98° C. (30 s), 35 cycles of 98° C. (10 s), 62° C. (20 s), 72° C. (2 min), and a final extension at 72° C. (2 min).

Next Generation Sequencing Indel formation at the TRAC locus was measured using Next Generation Sequencing (Illumina). Genomic PCR was performed according to the manufacturer's instructions using Q5® Hot Start polymerase (NEB); primers are listed in Table 1. Products were purified using SPRI cleanup with AMPure® XP beads (Beckman Coulter), and sequencing indices were added with a second round of PCR using indexing primers (Illumina), followed by a second SPRI cleanup. Samples were pooled and sequenced on an Illumina® MiniSeq according to the manufacturer's instructions. Analysis was performed using CRISPR RGEN.

Genome-wide, off-target analysis. Genomic DNA from human primary CD4⁺/CD8⁺ T cells was isolated using Gentra® Puregene® Kit (Qiagen) according to the manufacturer's instructions. CHANGE-seq was performed as described in the art. Briefly, purified genomic DNA was tagmented with a custom Tn5-transposome to an average length of 400 bp, followed by gap repair with Kapa HiFi™ HotStart Uracil+ DNA Polymerase (KAPA Biosystems) and Taq DNA ligase (NEB). Gap-repaired tagmented DNA was treated with USER enzyme (NEB) and T4 polynucleotide kinase (NEB). Intramolecular circularization of the DNA was performed with T4 DNA ligase (NEB) and residual linear DNA was degraded by a cocktail of exonucleases containing Plasmid-Safe™ ATP-dependent DNase (Lucigen), Lambda exonuclease (NEB) and Exonuclease I (NEB). In vitro cleavage reactions were performed with 125 ng of exonuclease-treated circularized DNA, 90 nM of SpCas9 protein (NEB), NEB buffer 3.1 (NEB) and 270 nM of sgRNA, in a 50 μL volume. Cleaved products were A-tailed, ligated with a hairpin adaptor (NEB), treated with USER enzyme (NEB) and amplified by PCR with barcoded universal primers NEBNext® Multiplex Oligos for Illumina® (NEB), using Kapa HiFi™ Polymerase (KAPA Biosystems). Libraries were quantified by qPCR (KAPA Biosystems) and sequenced with 151 bp paired-end reads on an Illumina® NextSeq™ instrument. CHANGE-seq data analyses were performed using open-source CHANGE-seq analysis software.

Cytokine Analysis. Cytokine analysis is performed using a V-PLEX® Proinflammatory Panel 1 Human Kit (MSD, Catalog No K15049D-2) according to the manufacturer's protocol. Measured cytokines include IFNγ, IL-1β, IL-2, IL-4, IL-6, IL-8, IL-10, IL-12p70, IL-13, and TNF-α. In brief, media was collected from the final day of cell culture before injection into mice and flash frozen and stored at −80° C. For co-culture samples, 250,000 T cells were co-cultured with 50,000 cancer cells in 250 μl ImmunoCult™ XF T cell expansion medium for 24 hours prior to media collection. On the day of the assay, media was thawed and 50 μl of media was used to perform all measurements in duplicate. Figures were produced using GraphPad PRISM 8. Data were normalized by calculating cytokine production per cell based on the total concentration of cells calculated at media collection.

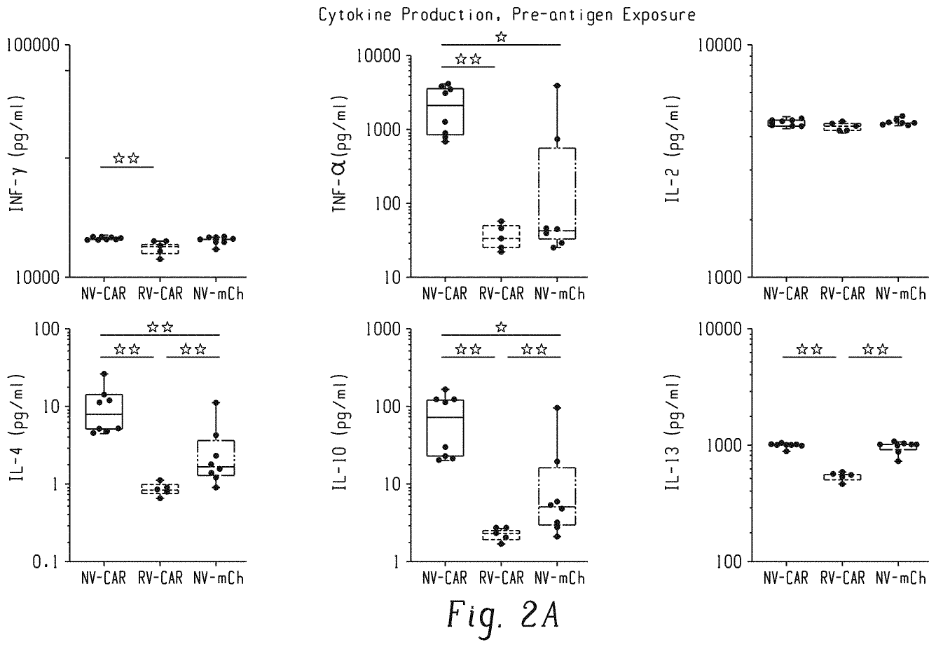

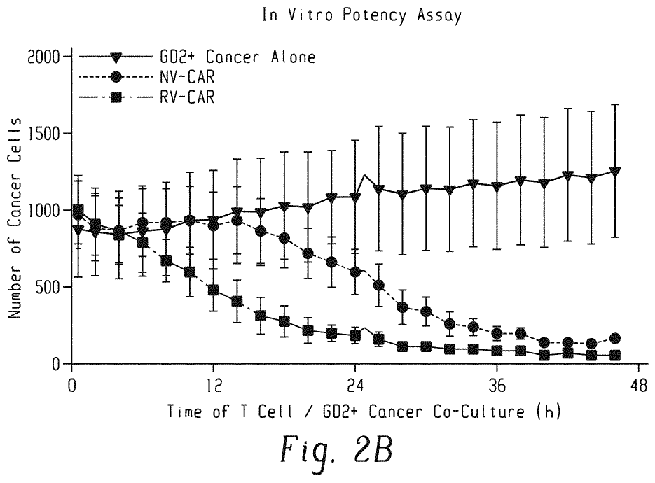

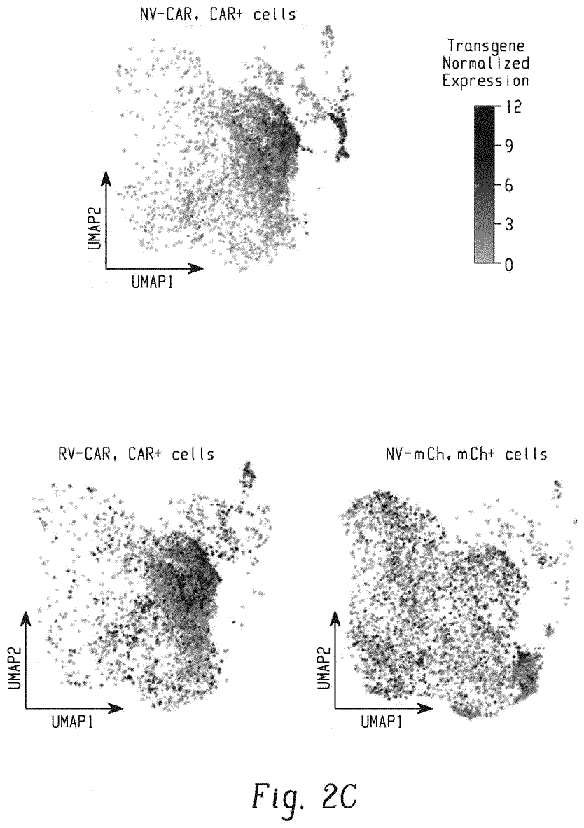

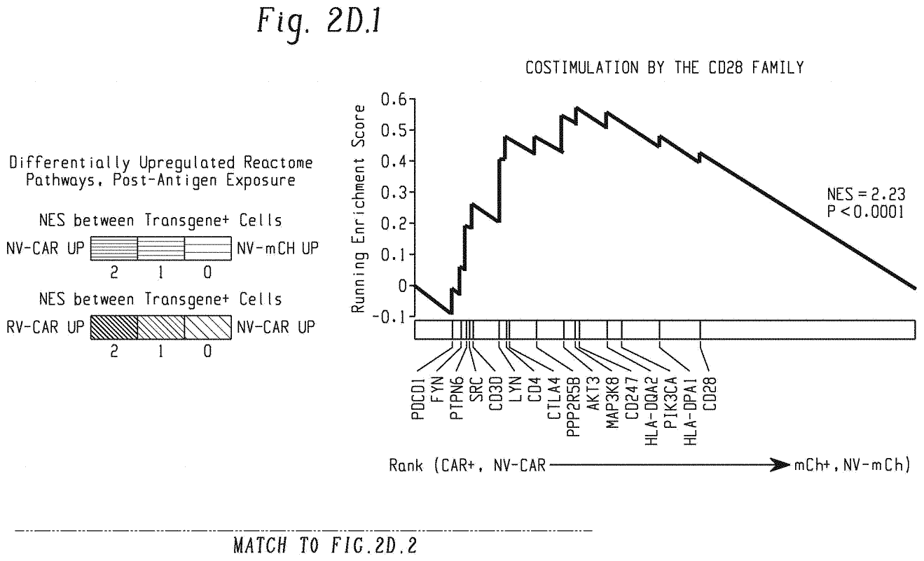

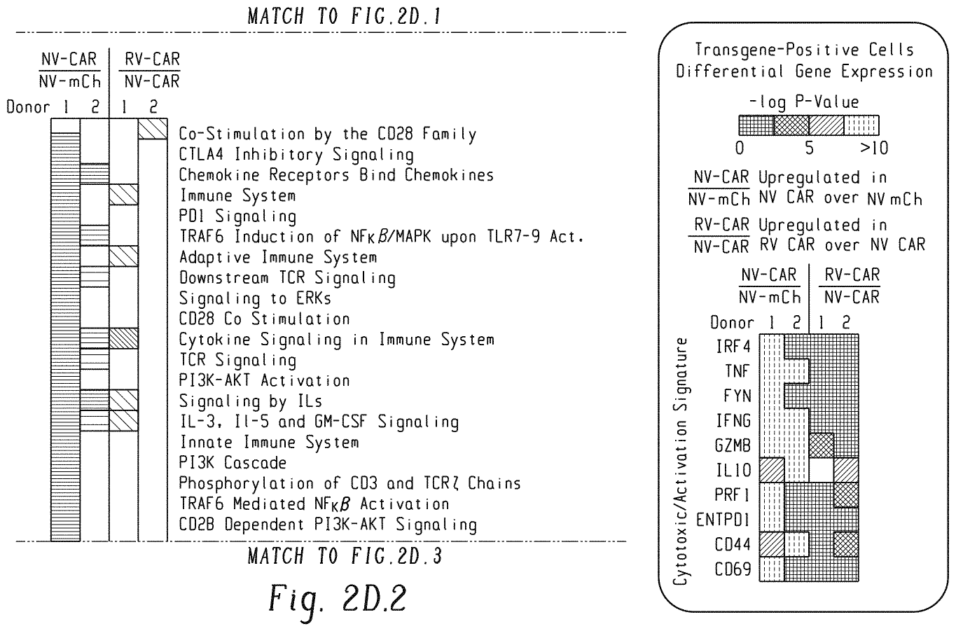

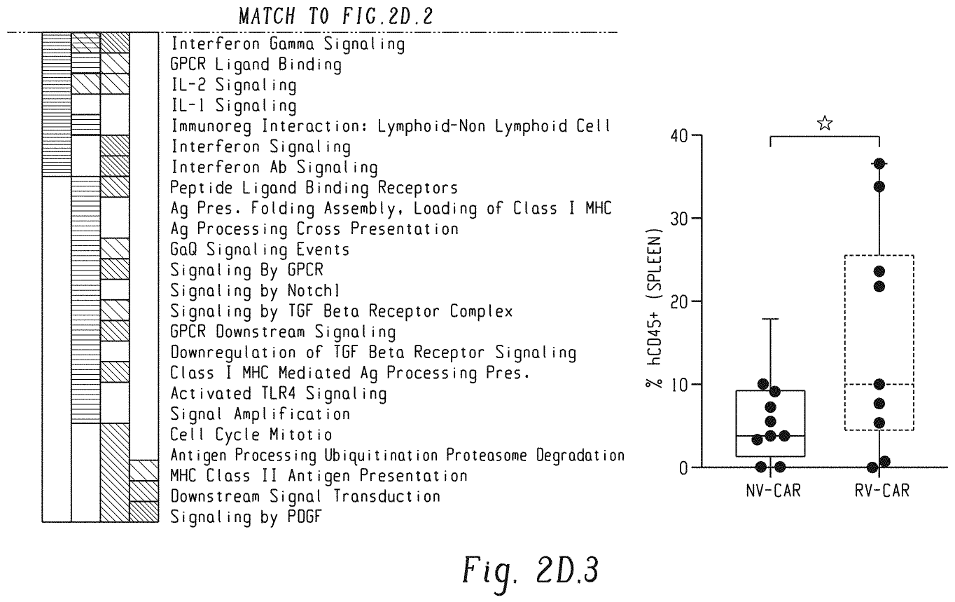

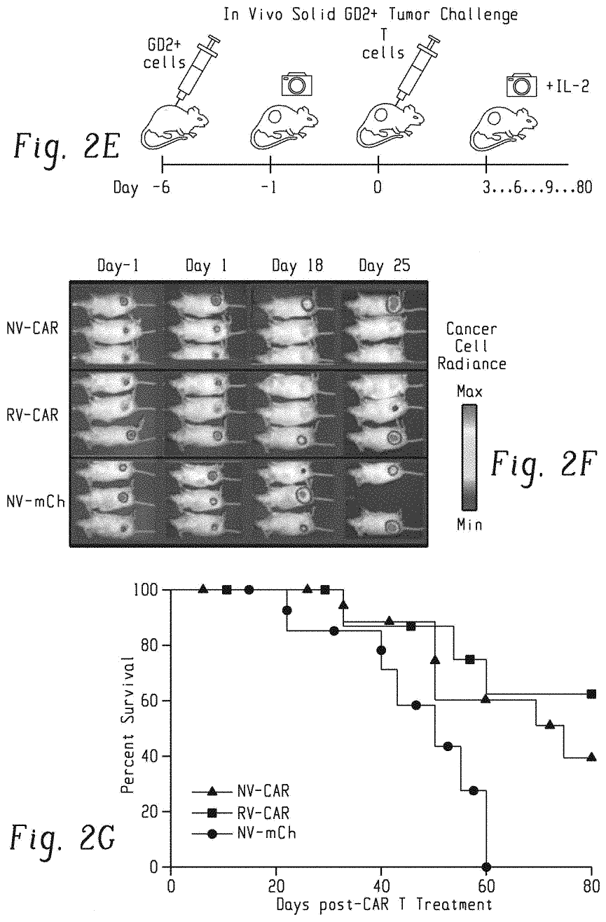

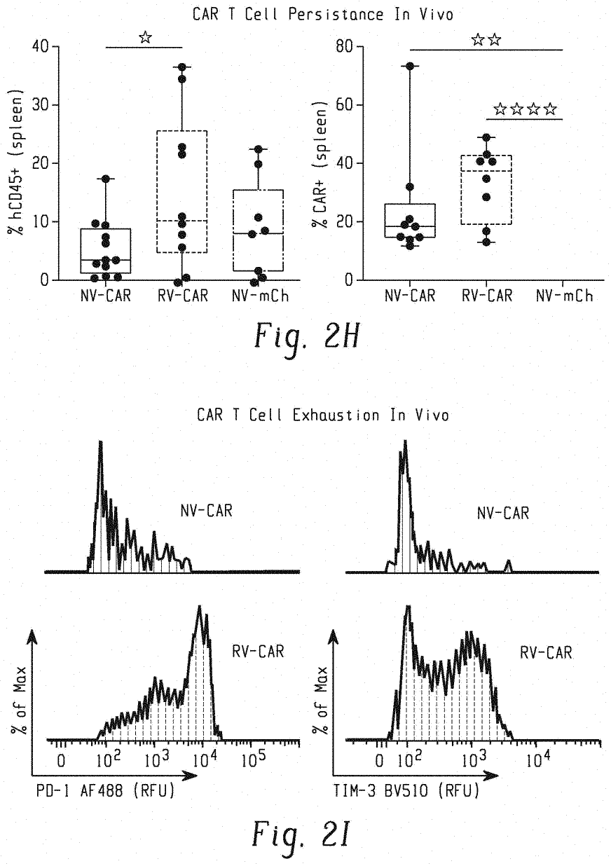

In Vitro Cytotoxicity Assays. For FIG. 2b: 10,000 AkaLUC-GFP CHLA20 cells were seeded in triplicate per condition in a 96 well flat bottom plate. 48 hours later, 50,000 T cells were added to each well. 1μl (0.05 μg) of CF® 594 Annexin V antibody (Biotium) was added to the wells. The plate was centrifuged at 100 g for 1 minute and then placed in the IncuCyte® S3 Live-Cell Analysis System (Sartorius, Catalog No 4647), stored at 37° C., 5% CO₂. Images were taken every 2 hours for 48 hours. Green object count was used to calculate the number of cancer cells in each well. Red object count was used to calculate the number of objects staining positive for Annexin V, an early apoptosis marker. Fluorescent images were analyzed with IncuCyte Base Analysis Software.

Single cell RNA sequencing: 24 hours prior to assay, 200,000 AkaLUC-CHLA-20 cells were plated in 12 well plates and cultured overnight. One week after electroporation (day 9 post-isolation), T cells were counted and pooled into a single bank for all characterization studies (scRNA-seq, IncuCyte® cytotoxicity assay and in vivo experiments). Media was aspirated from cancer cells, and 1 million T cells in ImmunoCult^{T}M-XF Medium +500 U/mL IL-2 were seeded on the cancer cells, then cultured for 24 hours. A parallel culture of T cells without cancer cells was set up at the same T cell density in a separate 12 well plate. The next day, co-culture cells were trypsinized for donor 1 and washed off the plate with media, and cells were singularized with a 35 μM cell strainer (Corning). For donor 2, co-culture cells were stained for CD45 and CAR, and FACS sorted into CD45⁺ CAR⁺ and CD45⁺CAR⁻ fractions prior to sample submission. Cells were counted with a Countess II FL cell counter using trypan blue exclusion (Thermo Fisher Scientific), and samples were prepared for single cell RNA sequencing with the 10× Genomics 3′ kit (v3 chemistry) according to the manufacturer's instructions. Libraries were sequenced using the Illumina® NovaSeq™ 6000 system. FASTQ files were aligned with Cellranger v3.1.0 to custom reference genomes that included added sequences for the transgene(s) used in each culture condition (e.g. the NV TRAC_CAR HDRT sequence, AkaLuc-GFP, etc.). Downstream analyses were performed using Seurat 3. For each sample, cells either expressing the transgene of interest (CAR or mCherry) were identified, and transgene-negative cells were removed from the dataset.

Gene set enrichment analysis (GSEA). GSEA was performed using the natural log-fold change values between sample pairs, using only the set of transgene-positive cells in each dataset. GSEA v.4.0.3 (Broad Institute) with the v7.1. Reactome signatures database from MSigDB was used with default parameters (1000 permutations). Data were exported and graphed in Microsoft Excel.

In vivo human neuroblastoma xenograft mouse model. All animal experiments were approved by the University of Wisconsin-Madison Animal Care and Use Committee (ACUC). Male and female NSG mice (9-25 weeks old) were subcutaneously injected with 10 million AkaLUC-GFP CHLA20 human neuroblastoma cells in the side flank to establish tumors. Six days later (Day 0), established tumors were verified by bioluminescence with the PerkinElmer In Vivo Imaging System (IVIS), and 10 million T cells were injected through the tail vein into each mouse. Mice were followed for weight loss and overall survival. On imaging days, mice were sedated using isoflurane and received intraperitoneal injections of ˜120 mg/kg D-luciferin (GoldBio). Fifteen minutes later, mice were imaged via IVIS. Imaging was repeated every 3 to 4 days, starting 1 day before initial T cell injection (Day -1). Mice were injected with 100,000 IU of human IL-2 subcutaneously on day 0, day 4, and with each subsequent IVIS reading. In order to quantify the total flux in the IVIS images, a region of interest (ROI) was drawn around the bottom half of each mouse with the total flux being calculated by Living Image® software (PerkinElmer; Total flux=the radiance (photons/sec) in each pixel summed or integrated over the ROI area (cm²)×4π). The absolute minimum total flux value was subtracted from each image to minimize background signal. For donors 1, 3, and 4, mice were maintained until tumors reached 20 mm in any dimension by digital caliper as defined by the ACUC.

Flow cytometric analysis of splenic and tumor-infiltrating T cells. For donor 2, all mice were euthanized on day 25. Tumors and spleens were removed, mechanically dissociated, and passed through a Corning® 35 μm cell strainer. Cell suspensions were centrifuged at 300 g for 10 minutes, and then digested with ACK lysing buffer (Lonza). The cells were then washed and centrifuged at 300 g for 10 minutes, and then resuspended in 10 ml PBS, 10 μl of which was added to 10 ml of ISOTON® diluent and counted on the COULTER COUNTER® Z1 Series Particle Counter (Beckman Coulter). From this count, 1×10⁶ cells were added to flow cytometry tubes in staining buffer (PBS with 2% FBS) and stained with antibodies for hCD45, mCD45, scFV 14G2a CAR, and PD-1 (see Table 1 for antibody information). The cells were then washed with PBS, centrifuged at 300 g for 10 minutes, and 0.5 μl of Ghost Dye™ Red 780 viability dye (Tonbo Biosciences) was added for 20 minutes at room temperature. Cells were then washed with staining buffer, spun down, and resuspended in 400 μl of staining buffer. Cells were then run on an Attune™ NXT flow cytometer (Thermo Fisher Scientific). Subsequent analysis was performed using Flowjo™ software (BD). For donors 3 and 4, spleens and tumors were analyzed as mice reached euthanasia criteria and were stained with an extended antibody panel outlined in Table 1.

Statistical analysis. Unless otherwise specified, all analyses were performed using GraphPad Prism (v.8.0.1), and error bars represent mean +-SD; ns=p>=0.05, * for p<0.05, ** for p<0.01, *** for p<0.001,**** for p<0.0001. For FIG. 2b, error bars show SEM. Statistical analyses for cytokine data (FIG. 1m, FIG. 2a, Extended Data FIG. 1e, Extended Data FIG. 2a) were performed using a two-tailed Mann-Whitney test in GraphPad Prism. All box plots show median (horizontal line), interquartile range (hinges), and smallest and largest values (whiskers). Statistical significance for differential gene expression was determined with Seurat 3 using the non-parametric Wilcoxon rank sum test. All 11 scRNA-seq samples were integrated and normalized, and 2 replicate samples per donor were combined to calculate differential expression between transgene-positive cells in each sample type. P values were adjusted using Bonferroni correction. p<0.001 was used as the threshold for assigning significant versus non-significant changes in gene expression. Volcano plots were generated in RStudio (v 1.1.456) using the ggplot2 and EnhancedVolcano packages. Statistical significance for FIG. 2h was calculated using the Mantel-Cox Test.

### Example 1: Design of HDR Donor Template Plasmid

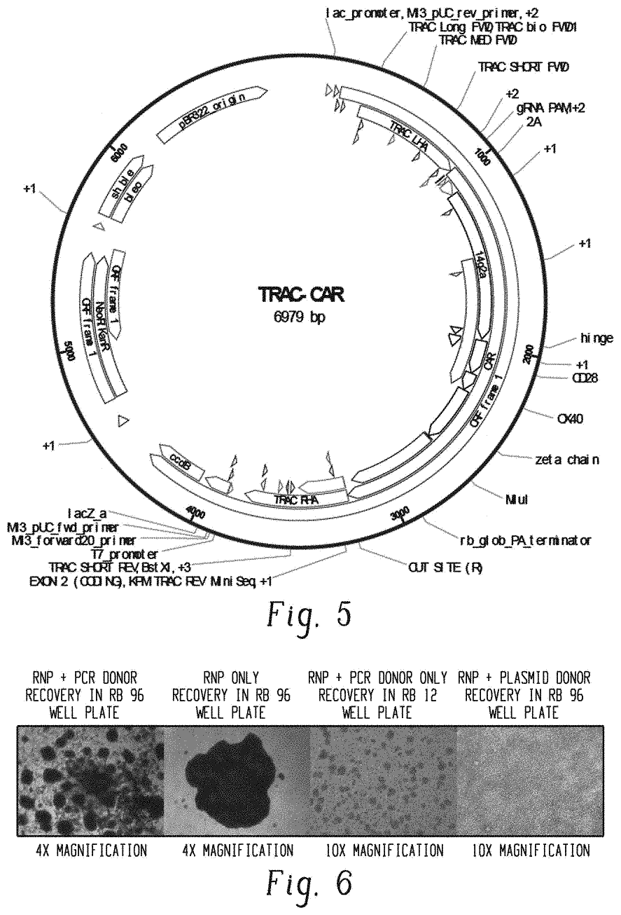

Described herein is a new method to insert CAR transgenes using Cas9 ribonucleoproteins (RNPs) targeted to a T-cell expressed gene locus such as the human TRAC locus in combination with a donor, specifically a PCR-amplified donor, encoding the CAR transgene (FIG. 1a). The TRAC exon is SEQ ID Nos. 13 and 14. For proof-of-principle, a published GD2-targeting CAR sequence was used for construction of the HDR donor template (HDRT). This HDRT is readily generated by PCR on a plasmid containing the CAR sequence. For the plasmid construction, a splice acceptor followed by a self-cleaving peptide, 2A, was cloned upstream of the GD2-CAR, and a transcriptional terminator followed by a poly A sequence was added downstream of the GD2-CAR. To facilitate HDR, homology arms around the Cas9 cut site in targeted gene (e.g., TRAC) was added to both ends of this construct. The resulting novel donor construct within a plasmid is shown in FIG. 5. The sequence of the TRAC-CAR is SEQ ID NO: 1.

### Example 2: Production of HDR Donor Template (HDRT)

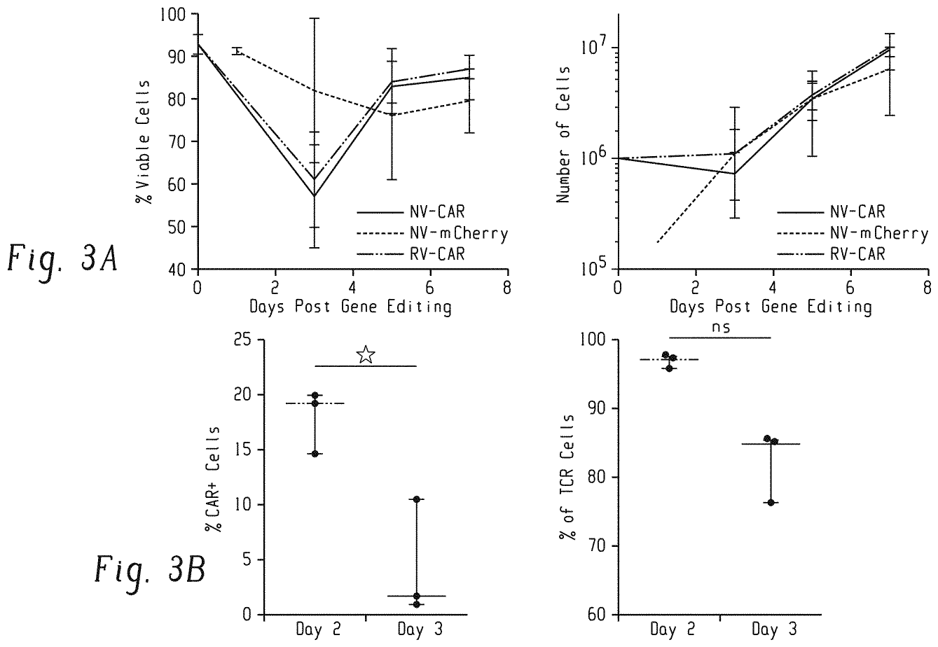

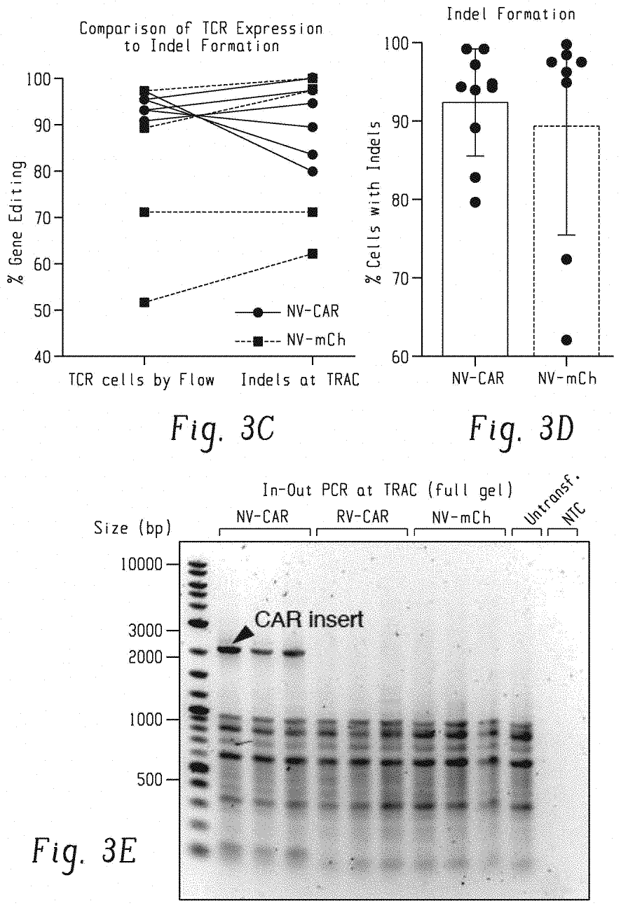

We next generated double-stranded DNA (dsDNA) HDR templates via PCR amplification off the plasmid and performed a two-step purification process to purify and concentrate the templates. Primary human T cells were electroporated with the HDR templates and Cas9 ribonucleoproteins (RNPs) targeting the human TRAC locus. Cells were subsequently expanded in xeno-free media and assayed on days 7 and 9 post-isolation (FIG. 1b). The viability of NV-CAR and RV-CAR T cells was comparably high (>80%) by the end of manufacturing (FIG. 3a). Cell proliferation and growth over 9 days was robust for both groups (FIG. 3a). We assessed gene editing at multiple points post-isolation and achieved higher levels of CAR integration when cells were edited at 48 hours after CD3/CD28/CD2 stimulation (FIG. 3b). As a control, we include an “NV-mCherry” (NV-mCh) condition in which cells harbor the same disruption of the TRAC locus, but with an insertion of a signaling-inert mCherry fluorescent protein in place of the CAR (FIG. 1b). Using these templates, we achieved consistently high genome editing rates across 31 technical replicates over 4 donors, with CAR integration averaging >15% as measured by flow cytometry (FIG. 1c, d). The mean fluorescence intensity (MFI) of CAR expression was significantly elevated and showed greater range (˜1.6 fold; FIG. 1d) in the RV-CAR samples in comparison to the NV-CAR samples indicating decreased CAR expression heterogeneity within the NV-CAR product and consistent with prior findings with CRISPR CAR T cells⁴. Within the NV-CAR samples, the TCR was knocked out in 90% of cells (FIG. 1c, e). We also assayed the immunophenotype by cell surface staining and found significantly elevated CD62L expression in both NV-CAR (CAR+TCR−fraction) and NV-mCh (mCh+TCR−fraction) cells relative to RV-CAR cells (CAR+TCR+fraction). The mean fluorescence intensity (MFI) of CD62L increased by ˜3 fold in the NV-CAR T cells relative to the RV-CAR T cells, indicating a naïve and/or stem cell memory or central memory phenotype in these populations after manufacturing (FIG. 1f).

### Example 3: NGS Sequencing and scRNA-seq

After obtaining high quality HDRT, next-generation sequencing of genomic DNA from the manufactured cell products confirmed high rates of indel formation at the TRAC locus, averaging 93.06% of reads for NV-CAR samples, and mirroring surface protein levels (FIG. 3c,d). Proper genomic integration of the CAR was confirmed via “in-out” PCR amplification with primers specific to the TRAC locus and the transgene (FIG. 3e). Highly sensitive genome-wide, off-target analysis for our editing strategy was assayed by CHANGE-seq. The top identified modified locus was the intended on-target site (FIG. 1h, i) with a rapid drop-off for off-target modifications elsewhere in the genome (Data not shown). The CHANGE-seq specificity ratio of our TRAC editing strategy is above average (0.056; 57th percentile) when compared to all editing strategies previously profiled by CHANGE-seq.

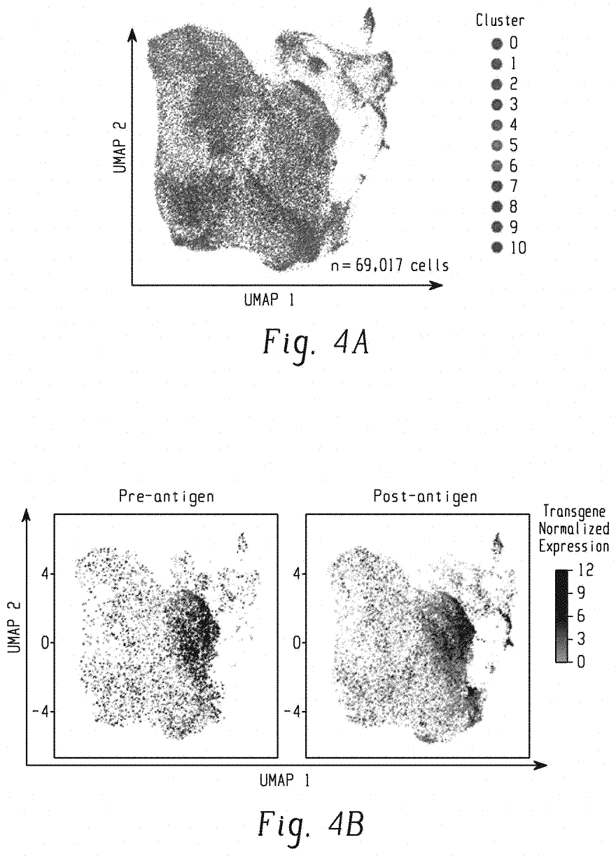

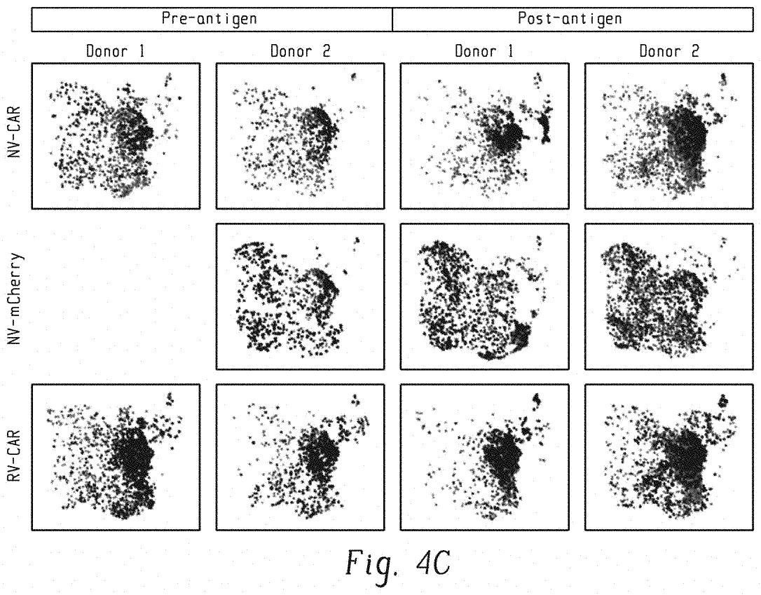

To further define the phenotypic differences between NV-CAR and RV-CAR T cells, we performed single-cell RNA-sequencing (scRNA-seq) on 29,122 cells from two different donors at the end of the manufacturing process (data not shown). To distinguish edited transgene-positive and transgene-negative cells within each sample, we aligned reads to a custom reference genome containing an added sequence mapping to the CAR or mCherry transgenes. We detected transgene expression in 6,376 across all samples assayed at the end of manufacturing (22% of assayed cells); and, all subsequent transcriptional analyses were carried out on transgene-positive cells only within each sample. UMAP dimensionality reduction of transgene-positive cells showed similar clustering for both NV-CAR and RV-CAR T cells but not NV-mCh T cells, indicating that the presence of CAR signaling alters the phenotype of the cells even prior to antigen stimulation (FIG. 1j, FIG. 4 a-c). We observed a variety of differentially expressed genes between both NV-CAR and RV-CAR T cells, and NV-CAR and NV-mCh T cells, which were significant for both donors (p<0.001 cutoff. Gene set enrichment analysis of the 6,209 differentially expressed genes (p<0.001 cutoff) between the CAR-positive T cells from the donor-matched NV-CAR and RV-CAR samples revealed enrichment of T cell activation and innate immune response pathways in the RV-CAR T cells (FIG. 1k; Data not shown), indicating that RV-CAR T cells activate broad signaling in response to the retroviral manufacturing process, CAR transgene, or retroviral vector elements. In comparison, none of these pathways were significantly enriched when comparing transgene-positive NV-CAR T cells to NV-mCh T cells (FIG. 1k). Transgene-positive RV-CAR T cells exhibited elevated levels of transcripts associated with an exhausted T cell signature (high CTLA4, ENTPD1, LAG3, TIGIT, CD244; FIG. 11) relative to transgene-positive NV-CAR T cells, but there were minimal significant differences in the exhaustion transcriptional profile between transgene-positive, donor-matched NV-CAR and NV-mCh T cells (Data not shown). Finally, we observed no significant changes in transcript levels for genes at or within 5 kb of off-target sites predicted by CHANGE-seq (Data not shown), indicating that any potential genomic disruptions at these sites did not lead to detectable changes in proximal transcripts.

### Example 4: Cytokine Production Levels

On day 9 of manufacturing, cytokine production levels were measured from the conditioned culture media. Prior to antigen exposure, RV-CAR T cells had higher levels of IFNγ, TNFα, IL-2, IL-4, IL-10, and IL-13, in comparison to both the NV-CAR and NV-mCh T cells (FIG. 1l). This result is consistent with the above transcriptional analysis showing hyperactive CAR signaling and recent observations that some RV-CAR T cells display elevated levels of tonic signaling prior to antigen exposure¹⁵. After a 24 h co-culture between the engineered T cells and GD2+ CHLA20 neuroblastoma, NV-CAR T cells either matched or surpassed the level of cytokine production of the RV-CAR T cells (FIG. 2a), indicating that NV-CART cells were capable of mounting a response to their target antigen, and suggesting that the RV-CAR T cells may be more exhausted prior to antigen exposure than the NV-CAR T cells. These trends, both pre-antigen exposure and post-antigen exposure, were also observed for IL-6, IL-1β and IL-12p70, but not for IL-8 (Data not shown)

### Example 5: In Vitro Potency of NV-CAR T Cells

After characterizing cellular phenotypes and gene expression at the end of the manufacturing process, we measured the in vitro potency of NV-CAR T cells against two GD2+ solid tumors: CHLA20 neuroblastoma and M21 melanoma (Data not shown). We observed robust killing using a 5:1 effector:target ratio for both NV-CAR and RV-CAR T cells (FIG. 2b, Data not shown). We again performed scRNA-seq on T cells that were co-cultured with CHLA20 neuroblastoma for 24 hours (FIG. 2c, data not shown). Gene set enrichment analysis of the 1,588 differentially expressed genes (p<0.001 cutoff) between the transgene-positive T cells from the NV-CAR and NV-mCh samples revealed high activation of T cell activation pathways in transgene-positive NV-CAR T cells (FIG. 2d), specifically CD28 activation pathways involving the CAR. When comparing the enrichment scores of pathways within CAR-positive cells between NV-CAR/RV-CAR paired samples, lower differences were observed in T cells post-antigen exposure relative to pre-antigen exposure (FIG. 2d vs. FIG. 1k; Data not shown). These results, corroborated by elevated cytokine production observed after CHLA20 co-culture (FIG. 2a), demonstrate that NV-CAR T cells can properly achieve high levels of activation upon antigen exposure, while avoiding potentially detrimental high tonic-signaling prior to antigen exposure. Tonic signaling is when intracellular signaling from both the TCR and CAR, in the absence of binding to the CAR target antigen, drives T cell phenotypes and differentiation toward effector or exhausted phenotypes given they both share common signaling pathways. Therefore, NV-CAR T cells that lack the TCR and have lower mean protein levels of the CAR could have lower intracellular tonic signaling, in the absence of binding to the CAR target antigen, relative to control viral CAR T cell products.

### Example 6: In Vivo Potency of NV-CART Cells

We assessed CAR T cell potency in vivo in an established human GD2+ neuroblastoma xenograft model. After 9 total days of culture, multiple replicate wells of RV-CAR, NV-CAR, or NV-mCh T cells were pooled for injection into NOD-SCID-γc^{-/-} (NSG) mice. Ten million T cells were delivered via tail vein injection to each NSG mouse with an established luciferase-expressing CHLA20 neuroblastoma tumor identified by bioluminescence (FIG. 2e). Tumor sizes were quantified over time by IVIS imaging and digital caliper (FIG. 2f). Both CAR-treated cohorts showed robust tumor regression in the first 3 weeks post-infusion (FIG. 2g, data not shown). These cohorts also showed significantly improved survival as compared to NV-mCh-treated mice; however, there was no significant difference in survival between NV-CAR and RV-CAR treated mice by day 80 (p-value=0.4099, n.s.). The percentage of CAR+ cells per dose was lower in NV-CAR T cells, which may have contributed to a slight decrease in complete remission rates (5/8 RV-CAR vs. 4/9 NV-CAR) but had no significant impact on overall survival, suggesting enhanced potency of the CAR-positive NV-CAR T cells. None of the control NV-mCh mice showed tumor regression, and all seven mice died of tumor progression by day 60. We also assessed persistence, memory and exhaustion phenotypes in T cells isolated from spleens and tumors. NV-CART cells persisted in both the spleens and tumors of the treated mice, but not for NV-mCh T cell treatments, indicating successful trafficking of NV-CAR T cells to the tumor microenvironment (FIG. 2h, Data not shown). Additionally, we observed that cells in the spleen had lower levels of PD-1 and TIM-3 exhaustion markers after NV-CAR treatment relative to the RV-CAR treatment (FIG. 2i), suggesting that the higher CAR MFI on RV-CARs (FIG. 1d) and detrimental signaling after expansion (FIG. 1k) could be contributing to increased propensity for exhaustion in RV-CARs. These findings demonstrate comparable potency of NV-CAR T cells to standard RV-CAR T cells, establishing the potential clinical relevance of NV-CAR T cells.

Example 7. Amplification of Long Double-stranded Homology-Directed Repair (HDR) Template

A set of primers was used to amplify a longer double-stranded HDR template from the donor template plasmid of SEQ ID NO: 1.

TABLE 4

| | | SEQ |
|---|---|---|
| Oligo | Sequence | ID NO: |
| TRAC Long Donor | TCGAGTAAACGGTAGTGCTGGG | 17 |
| FWD primer | | |
| TRAC Long Donor | CCTCTCCTGCCACCTTCTCTTC | 18 |
| REV primer | | |

This strategy generates a double-stranded HDR template length of 3.4 kb total, and the CAR knockin percentages have been consistently high as shown in FIG. 8. The leftmost homology arm includes 588 bp of the TRAC locus directly upstream of the cutsite, and the rightmost homology arm includes 499 bases. These homology arms are longer than those from Example 2 which were 383 bp (left) and 391 bp (right). It was unexpected that increasing the length of the homology arms would increase the percentage of CAR knockin about 2-fold compared to the templates with shorter homology arms.

### Discussion

Overall, we describe a rapid 9-day manufacturing of third generation GD2-specific CAR T cells using recombinant SpyCas9 protein and nucleic acids which results in stable, genomically-integrated, durable CAR expression (>80 days in vivo) without the use of any viral vectors. NV-CAR T cells exhibit proper TRAC-specific integration of the CAR transgene and an increased percentage and expression level of CD62L relative to conventional strategies. Robust upregulation of gene transcripts prevalent in cytotoxic transcriptional programs and secretion of pro-inflammatory cytokines like IFNγ and TNFα occur only after target antigen exposure, in contrast to conventional RV-CAR T cells that exhibit detrimental signaling during manufacturing. After injection into a GD2+ human neuroblastoma xenograft model, NV-CAR T cells induce strong regression of solid tumors compared to mock-edited T cells, and at levels comparable to RV-CAR T cells. NV-CAR T cells show reduced propensity to exhaustion at the gene expression and protein levels before antigen exposure, and at the protein level after antigen exposure.

Relative to conventional T cell manufacturing, our streamlined, nonviral manufacturing process could: 1) reduce the batch-to-batch variability, supply chain challenges, and costs associated with vector production alleviate a number of regulatory considerations (e.g., the need to monitor replication competency of the vector and the levels of xenogeneic components in the clinical cell product, notably plasmid DNA and serum during cell culture that can introduce infectious agents or toxic components); and 3) eliminate the potential for integration of viral elements into the human genome, which can generate a high degree of gene perturbation, up to 10⁴-10⁵ different insertional sites within a single product. Integration of the vector, in particular, presents risks of insertional oncogenesis, transgene silencing or overexpression, and adverse immune response to the vector, which could result in the rejection of therapeutic cells. While off-target analysis of genome editors is necessary for any clinical translation of our approach, there are now many experimental and computational tools that can readily be used for this purpose and next-generation high-fidelity Cas9 enzymes could be used to further decrease the potential for any off-target effects. Our fully-defined, nonviral manufacturing method therefore has high potential to enable the rapid and flexible manufacture of highly-defined and highly-potent CART cell products.

### Example 7: Analysis of NV-CRISPR CART Cells

FIG. 6 shows representative images of NV-CRISPR CART cells post-editing. Cells that demonstrate a high degree of aggregation are recovered at a higher rate. PCR donor refers to the HDRT. Aggregation can be indicative of successful genome editing and cell health, as non-aggregated cells are typically less viable with low to no levels of editing.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An ex vivo, non-viral method of site-specifically inserting a transgene containing a chimeric antigen receptor (CAR) gene into a T cell expressed gene to generate CAR T cells, comprising
preparing a non-viral double-stranded homology-directed repair (HDR) template comprising the transgene flanked by homology arms that are complementary to sequences on both sides of a cleavage site in the T cell expressed gene,
introducing into a population of unmodified T cells a Cas9 ribonucleoprotein (RNP) and the double-stranded HDR template, to provide the CAR T cells
wherein the Cas9 RNP comprises a Cas9 protein and a guide RNA that directs double stranded DNA cleavage of a cleavage site in the T cell expressed gene,
wherein the non-viral double-stranded HDR template contains the transgene sequence flanked by homology arms that are complementary to sequences on both sides of the cleavage site in the T cell expressed gene, and
wherein the transgene is specifically integrated into the cleavage site of the T cell expressed gene locus created by the Cas9 RNP in the CAR T cells, and
culturing the CAR T cells in xeno-free medium to provide a cultured population of CAR T cells having the transgene specifically integrated in the T cell expressed gene,
wherein, in the cultured population of CAR T cells, an endogenous promoter of the T cell expressed gene drives expression of the transgene, or wherein the transgene includes a promoter that drives expression of the transgene, and
wherein the CAR gene encodes a fusion protein comprising one or more antigen-specific extracellular domains coupled to an intracellular domain by a transmembrane domain.

2. The method of claim 1, wherein the homology arms have a length of 400 to 1000 base pairs.

3. The method of claim 1, wherein the homology arms have a length of 450 to 750 base pairs.

4. The method of claim 1, wherein the antigen-specific extracellular domain of the CAR is from antigen recognition molecule that recognizes a cell surface molecule on malignant cells such as hematologic malignancies or solid tumors.

5. The method of claim 1, wherein the T cell expressed gene is TRAC, TRBC, AAVS1, TET2, FAS, BID, CTLA4, PDCD1, CBLB, PTPN6, CIITA or B2M genes.

6. The method of claim 1, wherein the CART cell kills target-antigen-positive human cancer cells in vitro in a co-culture assay, in an in vivo animal model, or both.

7. The method of claim 1, wherein the CAR T cells have activity against an antigen on a solid tumor in vitro or in vivo.

8. The method of claim 1, further comprising imaging the population of CAR T cells and determining the degree of aggregation of the CAR T cells, and optionally selecting a population of aggregated CAR T cells.

9. The method of claim 1, further comprising, prior to introducing the double-stranded HDR template, determining the concentration and purity of the double-stranded HDR template, wherein the double-stranded HDR template has an OD260/OD280 of 1.8 to 2.1, and/or an OD260/OD230 of 2.0 to 2.3, and diluting the double-stranded HDR template to a concentration of 2000 to 10000 ng/μ1.

10. The method of claim 1, wherein the intracellular domain comprises a CD28, ICOS, CD27, 4-1BB, OX40, CD40L, or CD3-ζ intracellular domain and the transmembrane domain comprises a CD4, CD8α, CD28, or CD3-ζ transmembrane domain.

11. The method of claim 1, wherein the HDR template comprises a coding sequence for a fluorescent protein, a synthetic receptor, a gene for a cytokine signaling protein, or a short hairpin (sh)RNA.

12. The method of claim 1, wherein the non-viral double-stranded HDR template sequentially comprises a left homology arm—a splice acceptor site—a self-cleaving peptide sequence—CAR gene—a polyA terminator—a right homology arm.

13. The method of claim 12, wherein the self-cleaving peptide sequence is a T2A coding sequence.

14. The method of claim 1, wherein the double-stranded HDR template is produced by amplifying a sequence from SEQ ID NO: 1.

15. The method of claim 14, wherein the forward primer comprises SEQ ID NO 17 and the reverse primer comprises SEQ ID NO: 18.

16. The method of claim 1, wherein the guide RNA targets the 5′ end of the first exon of TRAC.

17. The method of claim 16, wherein the guide RNA comprises SEQ ID NOs: 2 and 3.

18. The method of claim 1, wherein culturing is done in round bottom culture wells at 20% of standard culture volume for the wells.

19. The method of claim 1, wherein the unmodified T cells are autologous T cells isolated from a patient in need of cancer treatment, or T cells from an allogeneic healthy donor.

20. The method of claim 1, wherein more than 4% of the population of unmodified T cells has the CAR transgene inserted into their genomes and expressed on the cell surface.

21. A non-viral produced CAR T cell with a genome having a CAR sequence specifically integrated into a T cell expressed gene, wherein the T cell is enriched for the CD62L and/or CD45RA markers indicative of naïve and stem cell memory phenotypes compared to retroviral-produced control CAR T cells.

22. The non-viral produced CART cell of claim 21, wherein the CD62L and/or CD45RA markers are enriched more than 2-fold compared to the CD62L and/or CD45RA markers compared to retroviral-produced control CAR T cells.

23. A non-viral produced CAR T cell with a genome having a CAR sequence specifically integrated into a T cell expressed gene, wherein the T cell has reduced expression of TIM3 and/or LAG3 markers of T cell exhaustion compared to retroviral-produced control CAR T cells.

24. The non-viral produced CAR T cell of claim 23, wherein the TIM3 and/or LAG3 markers are reduced more than 2-fold compared to the TIM3 and/or LAG3 markers compared to retroviral-produced control CAR T cells.

25. A plasmid of SEQ ID NO: 1.


SEQUENCE LISTING (18 sequences)

SEQ ID NO: 1 - DNA, 6979 bp, Artificial Sequence
  TRAC-CAR
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttgagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacaagcttctgacctcttctcttcctcccacagggcctcgagagatctggcagcggagagggcagaggaagtcttctaacatgcggtgacgtggaggagaatcccggccctaggctcgagatggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccagacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - DNA, 58 bp, Artificial Sequence
  CAR genetic construct sense strand
tcctaaccctgatcctcttgtcccacagatatccagaaccctgaccctgccctgtacc

SEQ ID NO: 3 - DNA, 58 bp, Artificial Sequence
  CAR genetic construct antisense strand
aggattgggactaggagaacagggtgtctataggtcttgggactgggacggcacatgg

SEQ ID NO: 4 - DNA, 22 bp, Artificial Sequence
  target sequence
  misc_feature (20)..(20): n is a, c, g, or t
cagggttctggatatctgtngg

SEQ ID NO: 5 - protein, 1368 aa, Streptococcus pyogenes [sequence omitted]

SEQ ID NO: 6 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 7 - protein, 1082 aa, Neisseria
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 8 - protein, 1395 aa, Treponema
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 9 - DNA, 19 bp, Artificial Sequence
  TRAC gRNA
cagggttctggatatctgt

SEQ ID NO: 10 - DNA, 35 bp, Artificial Sequence
  cr RNA
cagggttctggatatctgtgttttagagctatgct

SEQ ID NO: 11 - DNA, 22 bp, Artificial Sequence
  Primer
cctttttcccatgcctgccttt

SEQ ID NO: 12 - DNA, 22 bp, Artificial Sequence
  Primer
taaggccgagaccaccaatcag

SEQ ID NO: 13 - DNA, 33 bp, Artificial Sequence
  Primer
acactctttccctacacgacgctcttccgatct

SEQ ID NO: 14 - DNA, 34 bp, Artificial Sequence
  Primer
gtgactggagttcagacgtgtgctcttccgatct

SEQ ID NO: 15 - DNA, 22 bp, Artificial Sequence
  Primer
atcttgtgcgcatgtgaggggc

SEQ ID NO: 16 - DNA, 22 bp, Artificial Sequence
  Primer
gcaagccaggactccaccaacc

SEQ ID NO: 17 - DNA, 22 bp, Artificial Sequence
  TRAC Long Donor FWD primer
tcgagtaaacggtagtgctggg

SEQ ID NO: 18 - DNA, 22 bp, Artificial Sequence
  TRAC Long Donor REV primer
cctctcctgccaccttctcttc